(19) United States
(12) Patent Application Publication
(10) Pub. No.: US 2024/0246567 A1
(43) Pub. Date: Jul. 25, 2024

(54) VEHICLE CONTROL DEVICE AND VEHICLE CONTROL METHOD

(71) Applicant: DENSO CORPORATION, Kariya-city (JP)

(72) Inventors: Takuya KUME, Kariya-city (JP); Kazuki Izumi, Kariya-city (JP)

(21) Appl. No.: 18/624,969

(22) Filed: Apr. 2, 2024

Related U.S. Application Data
(63) Continuation of application No. PCT/JP2022/035813, filed on Sep. 27, 2022.

(30) Foreign Application Priority Data
Oct. 5, 2021 (JP) 2021-164187
Sep. 1, 2022 (JP) 2022-139518

(51) Int. Cl. B60W 60/00; A61B 5/00; A61B 5/18; B60W 30/18; B60W 40/08; B60W 50/14

(52) U.S. Cl. CPC B60W 60/00133; A61B 5/18; A61B 5/4809; A61B 5/6893; B60W 30/18163; B60W 40/08; B60W 50/14; B60W 2040/0827; B60W 2540/229

(57) ABSTRACT

A vehicle control device that performs sleep-permitted automated driving during which a driver is permitted to sleep is configured to estimate a condition of the driver and to exercise control to reduce stimulation to the driver when it is estimated that the driver is in a sleep state during the sleep-permitted automated driving of the vehicle.

### CROSS REFERENCE OF RELATED APPLICATIONS

The present application is a continuation application of International Patent Application No. PCT/JP2022/035813 filed on Sep. 27, 2022 which designated the U.S. and claims the benefit of priority from Japanese Patent Application No. 2021-164187 filed on Oct. 5, 2021 and Japanese Patent Application No. 2022-139518 filed on Sep. 1, 2022. The entire disclosures of all of the above applications are incorporated herein by reference.

### TECHNICAL FIELD

The present disclosure relates to a control device for a vehicle and a control method for a vehicle.

### BACKGROUND

A related art discloses a control unit for automated driving having automated driving functions of Level 1 to Level 5 in addition to a manual driving function of Level 0.

### SUMMARY

A vehicle control device that performs sleep-permitted automated driving during which a driver is permitted to sleep is configured to estimate a condition of the driver and to exercise control to reduce stimulation to the driver when it is estimated that the driver is in a sleep state during the sleep-permitted automated driving of the vehicle.

### BRIEF DESCRIPTION OF DRAWINGS

FIG. 1 is a drawing illustrating an example of a general configuration of a vehicular system;

FIG. 2 is a drawing illustrating an example of a general configuration of an automated driving ECU;

FIG. 3 is a flowchart showing an example of a flow of stimulation reduction related processing at an automated driving ECU;

FIG. 4 is a drawing illustrating an example of a general configuration of a vehicular system;

FIG. 5 is a drawing illustrating an example of a general configuration of an automated driving ECU;

FIG. 6 is a flowchart showing an example of a flow of stimulation reduction related processing at an automated driving ECU;

FIG. 7 is a drawing illustrating an example of a general configuration of a vehicular system;

FIG. 8 is a drawing illustrating an example of a general configuration of an automated driving ECU;

FIG. 9 is a flowchart showing an example of a flow of stimulation reduction related processing at an automated driving ECU;

FIG. 10 is a drawing illustrating an example of a general configuration of a vehicular system;

FIG. 11 is a drawing illustrating an example of a general configuration of an automated driving ECU;

FIG. 12 is a drawing illustrating an example of a general configuration of a vehicular system;

FIG. 13 is a flowchart showing an example of a flow of stimulation reduction related processing at an automated driving ECU;

FIG. 14 is a drawing illustrating an example of a general configuration of a vehicular system;

FIG. 15 is a drawing illustrating an example of a general configuration of an automated driving ECU;

FIG. 16 is a drawing illustrating an example of a general configuration of a vehicular system;

FIG. 17 is a drawing illustrating an example of a general configuration of an automated driving ECU;

FIG. 18 is a drawing explaining two times of lane change for overtaking;

FIG. 19 is a drawing illustrating an example of a general configuration of a vehicular system;

FIG. 20 is a drawing illustrating an example of a general configuration of an automated driving ECU;

FIG. 21 is a drawing illustrating an example of a general configuration of a vehicular system; and

FIG. 22 is a drawing illustrating an example of a general configuration of an automated driving ECU.

### DETAILED DESCRIPTION

As an automation level, for example, an automation level divided into Levels 0 to 5, defined by SAE, is known. Level 0 is a level at which a driver performs all the driving tasks without intervention of a system. Level 0 is equivalent to so-called manual driving. Level 1 is a level at which a system assists either steering or acceleration/deceleration. Level 2 is a level at which a system assists both steering and acceleration/deceleration. The automated driving of Levels 1 to 2 is automated driving during which a driver has an obligation to do monitoring related to safe driving (hereafter, simply referred to as monitoring obligation). Level 3 is a level at which a system can perform all the driving tasks in such a specific place as a highway and a driver performs a driving operation in emergency. Level 4 is a level at which a system can perform all the driving tasks except on a road the system cannot cope with and in such specific situations as an extreme environment. Level 5 is a level at which a system can perform all the driving tasks in every environment. The automated driving of Level 3 or higher levels is automated driving during which a driver does not have a monitoring obligation. The automated driving of Level 4 or higher level is automated driving during which a driver is permitted to sleep.

A related art discloses a technology for perform automated driving of Level 4 or high level but is not on the assumption that control is made to differ depending on whether a driver is during sleep or wakefulness. Unlike during wakefulness, it is presumed that a driver desires that his/her sleep is not disturbed during sleep. In the technology disclosed in the related art, control cannot be implemented according to whether a driver is during sleep or wakefulness and thus the driver's convenience can be degraded.

The present disclosure provides a vehicle control device and a vehicle control method with which a driver's convenience can be enhanced during automated driving during which the driver is permitted to sleep.

According to one aspect of the present disclosure, a vehicle control device used in a vehicle that performs sleep-permitted automated driving during which a driver is permitted to sleep is provided. The vehicle control device includes: a driver condition estimation unit that is configured to estimate a condition of the driver; and a stimulation reduction control unit that is configured to exercise control to reduce stimulation to the driver when the driver condition estimation unit estimates that the driver is in a sleep state during the sleep-permitted automated driving of the vehicle.

According to one aspect of the present disclosure, a vehicle control method that is be used in a vehicle that performs sleep-permitted automated driving during which a driver is permitted to sleep is provided. The control method is performed by at least one processor. The control method includes: a driver condition estimation step of estimating a condition of the driver; and a stimulation reduction control step of, when it is estimated at the driver condition estimation step that the driver is in a sleep state during the sleep-permitted automated driving of the vehicle, exercising control to reduce stimulation to the driver.

According to the above-mentioned configuration, when it is estimated during sleep-permitted automated driving that a driver is in a sleep state, control is implemented to reduce stimulation to the driver; therefore, when a driver is in a sleep state during sleep-permitted driving, stimulation to the driver can be suppressed from disturbing the sleep. As a result, during automated driving during which a driver is permitted to sleep, the driver's convenience can be further enhanced.

### First Embodiment

### <General Configuration of Vehicular System 1>

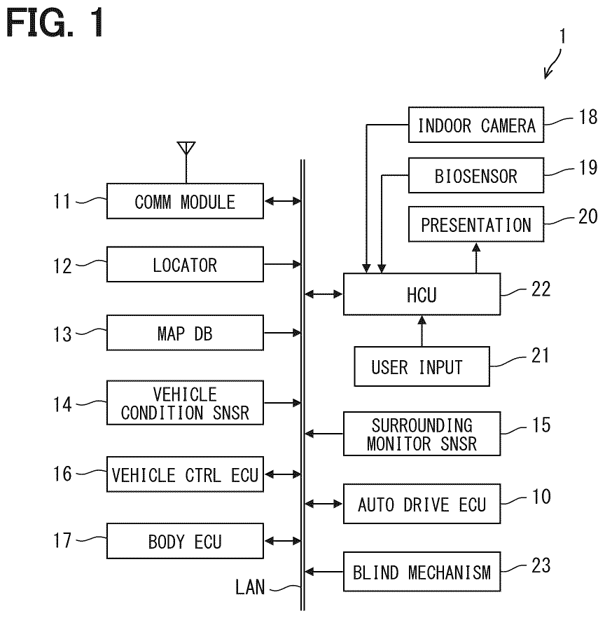

Hereafter, a description will be given to a first embodiment of the present disclosure with reference to the drawings. The vehicular system 1 shown in FIG. 1 can be used in a vehicle capable of automated driving (hereafter, referred to as an automated driving vehicle). As shown in FIG. 1, the vehicular system 1 includes an automated driving ECU 10, a communication module 11, a locator 12, a map database (hereafter, referred to as map DB) 13, a vehicle condition sensor 14, a surroundings monitoring sensor 15, a vehicle control ECU 16, a body ECU 17, an interior camera 18, a biosensor 19, a presentation device 20, a user input device 21, HCU (Human Machine Interface Control Unit) 22, and a blind mechanism 23. For example, the vehicular system can be so configured that the automated driving ECU 10, the communication module 11, the locator 12, the map DB 13, the vehicle condition sensor 14, the surroundings monitoring sensor 15, the vehicle control ECU 16, the body ECU 17, HCU 22, and the blind mechanism 23 are connected with an in-vehicle LAN (refer to LAN in FIG. 1). A vehicle using the vehicular system 1 need not be an automobile but in the following description, a case where the vehicular system is used in an automobile will be taken as an example.

As the stages of automated driving of an automated driving vehicle (hereafter, referred to as automation levels), a plurality of levels can exist as defined by SAE, for example. The automation level can be divided, for example, into LVs 0 to 5 as described below:

LV 0 is a level at which a driver performs all the driving tasks without intervention of a system. The driving task may be rephrased to dynamic driving task. Examples of the driving tasks include, for example, steering, acceleration/deceleration, and surroundings monitoring. LV 0 is equivalent to so-called manual driving. LV 1 is a level at which a system assists either steering or acceleration/deceleration. LV 1 is equivalent to so-called manual driving. LV 2 is a level at which a system assists both steering and acceleration/deceleration. LV 2 is equivalent to so-called partial driving automation. LVs 1 to 2 are also defined as part of automated driving.

For example, automated driving of LV 1 to 2 is automated driving during which a driver has an obligation to do monitoring related to safe driving (hereafter, simply referred to as monitoring obligation). That is, the automated driving of LVs 1 to 2 is equivalent to monitoring obliged automated driving. An example of monitoring obligation is visual surroundings monitoring. The automated driving of LVs 1 to 2 can be rephrased to second task prohibited automated driving. The second task is an action other than driving permitted to a driver and is a predetermined specific action. The second task can also be rephrased to secondary activity, other activity, or the like. The second task should not prevent a driver from coping with a driving operation handover request from an automated driving system. Assumed examples of second tasks include viewing of such contents as videos, operation of a smartphone or the like, such actions as reading and taking a meal.

The automated driving of LV 3 is at a level at which a system can perform all the driving tasks under a specific condition and a driver performs driving operation in emergency. When a driving change is requested from a system during automated driving of LV 3, a driver is required to be capable of swiftly coping therewith. This driving change can also be rephrased to a transfer of a surroundings monitoring obligation from a vehicle-side system to a driver. LV 3 is equivalent to so-called conditional driving automation. LV 3 includes an area limited LV 3 at which automated driving is limited to a specific area. Highway can be included in the specific area cited here. The specific area may be, for example, a specific lane. Another example of LV 3 is a congestion limited LV 3 at which automated driving is limited to a time of congestion. The congestion limited LV 3 can be so configured that automated driving is limited to, for example, a time of congestion on a highway. An automobile road may be included in the highway.

The automated driving of LV 4 is at a level at which a system can all the driving tasks except on a road the system cannot cope with and in such specific situations as an extreme environment. LV 4 is equivalent to so-called high driving automation. The automated driving of LV 5 is at a level at which a system can perform all the driving tasks in every environment. LV 5 is equivalent to so-called full driving automation. The automated driving of LV 4 and LV 5 can be performed, for example, in a traveling section for which highly accurate map data has been prepared. The highly accurate map data will be described later.

For example, the automated driving of LVs 3 to 5 is defined as automated driving during which a driver does not have a monitoring obligation. That is, the automated driving of LVs 3 to 5 is equivalent to automated driving free from a monitoring obligation. The automated driving of LVs 3 to 5 can be rephrased to second task permitted automated driving. Of the automated driving of LVs 3 to 5, the automated driving of LV 4 or higher level is equivalent to automated driving during which a driver is permitted to sleep. That is, the automated driving of LV 4 or higher level is equivalent to sleep-permitted automated driving. Of the automated driving of LV 3 to 5, the automated driving of Level 3 is equivalent to automated driving during which a driver is not permitted to sleep. In an automated driving vehicle according to the present embodiment, an automation level is switchable. The present embodiment may be so configured that only some of LVs 0 to 5 is switchable. In an automated driving vehicle according to the present embodiment, at least sleep-permitted automated driving can be performed.

The communication module 11 sends and receives information to and from a center external to the subject vehicle by radiocommunication. That is, the communication module performs wide area communication. The communication module 11 receives traffic jam information and the like from the center by wide area communication. The communication module 11 may send and receive information to and from another car by radiocommunication. That is, the communication module may perform inter-vehicle communication. The communication module 11 may send and receive information to and from a roadside device installed on the roadside by radiocommunication. That is, the communication module may perform vehicle roadside communication. To perform vehicle roadside communication, the communication module 11 may receive information of a nearby vehicle of the subject vehicle sent from the nearby vehicle through a roadside device. The communication module 11 may receive information of a nearby vehicle of the subject vehicle sent from the nearby vehicle by wide area communication through a center.

The locator 12 includes a GNSS (Global Navigation Satellite System) receiver and an inertia sensor. The GNSS receiver receives a positioning signal from a plurality of positioning satellites. The inertia sensor includes, for example, a gyro sensor and an acceleration sensor. The locator 12 combines a positioning signal received by the GNSS receiver and a measurement result from the inertia sensor and thereby successively potions a vehicle position of the subject vehicle mounted with the locator 12 (hereafter, referred to as subject vehicle position). The subject vehicle position can be expressed by, for example, coordinates of latitude and longitude. To position a subject vehicle position, the present embodiment may be so configured as to use a mileage as well determined from a signal successively outputted from a vehicle speed sensor mounted in the vehicle.

The map DB 13 is a nonvolatile memory and holds highly accurate map data. The highly accurate map data is map data more accurate than map data used in route guidance in a navigation function. The map DB 13 may hold map data used in route guidance as well. The highly accurate map data includes information usable in automated driving, for example, three-dimensional shape information of a road, number of lanes information, information indicating a traveling direction permitted for each lane. In addition, the highly accurate map data may also include, for example, information of node points indicating the positions of both ends with respect to such a road marking as a lane marking. The locator 12 may so configured as to use three-dimensional shape information of a road, not to use the GNSS receiver. For example, the locator 12 may be so configured as to use three-dimensional shape information of a road and a detection result from LIDAR (Light Detection and Ranging/Laser Imaging Detection and Ranging), which detects a point group of feature points of a road shape and a structure, or such a surroundings monitoring sensor 15 as a surroundings monitoring camera to identify a subject vehicle position. Three-dimensional shape information of a road may be generated based on a captured image by REM (Road Experience Management).

Map data distributed from an external server may be received by wide area communication through the communication module 11 and be stored in the map DB 13. In this case, the present embodiment may be so configured that a volatile memory is used for the map DB 13 and the communication module 11 successively acquires map data of an area corresponding to a subject vehicle position.

The vehicle condition sensor 14 is a sensor group for detecting various statuses of the subject vehicle. The vehicle condition sensor 14 includes the vehicle speed sensor, a steering torque sensor, an accelerator sensor, a brake sensor, and the like. The vehicle speed sensor detects a speed of the subject vehicle. The steering torque sensor detects a steering torque applied to a steering wheel. The accelerator sensor detects whether an accelerator pedal has been depressed. For the accelerator sensor, an accelerator effort sensor that detects pedal effort applied to the accelerator pedal can be used. For the accelerator sensor, an accelerator stroke sensor that detects a depression amount of the accelerator pedal may be used. For the accelerator sensor, an accelerator switch that outputs a signal corresponding to whether the accelerator pedal has been depressed may be used. The brake sensor detects whether a brake pedal has been depressed. For the brake sensor, a braking effort sensor that detects pedal effort applied to the brake pedal can be used. For the brake sensor, a brake stroke sensor that detects a depression amount of the brake pedal may be used. For the brake sensor, a brake switch that outputs a signal corresponding to whether the brake pedal has been depressed may be used. The vehicle condition sensor 14 outputs detected sensing information to the in-vehicle LAN. The present embodiment may be so configured that sensing information detected by the vehicle condition sensor 14 is outputted to the in-vehicle LAN through ECU mounted in the subject vehicle.

The surroundings monitoring sensor 15 monitors an environment surrounding the subject vehicle. For the example, the surroundings monitoring sensor 15 detects such an obstacle surrounding the subject vehicle as a pedestrian, such a moving object as another vehicle, and such a stationary object as a falling object on a road. In addition, the surroundings monitoring sensor detects such a road marking as a traveling lane marking surrounding the subject vehicle. The surroundings monitoring sensor 15 is, for example, a surroundings monitoring camera that picks up an image of a predetermined range surrounding the subject vehicle, or such a sensor as a millimeter wave radar, a sonar, LIDAR, or the like that sends a prospecting wave to a predetermined range surrounding the subject vehicle. The predetermined range may be a range that at least partially includes the front, rear, left and right of the subject vehicle. The surroundings monitoring camera successively outputs a captured image successively picked up to the automated driving ECU 10 as sensing information. Such a sensor as a sonar, a millimeter wave radar, LIDAR, or the like that sends a prospecting wave successively outputs a scanning result based on a reception signal obtained when a reflected wave reflected by an obstacle is received to the automated driving ECU 10 as sensing information. The present embodiment may be so configured that sensing information detected by the surroundings monitoring sensor 15 is outputted to the automated driving ECU 10 without intervention of the in-vehicle LAN.

The vehicle control ECU 16 is an electronic control unit that controls driving of the subject vehicle. The driving control includes acceleration/deceleration control and/or steering control. The vehicle control ECU 16 includes a steering ECU that exercises steering control, a power unit control ECU that exercises acceleration/deceleration control, a brake ECU, and the like. The vehicle control ECU 16 outputs a control signal to each of driving control devices, such as an electronically controlled throttle, a brake actuator, and an EPS (Electric Power Steering) motor, and the like mounted in the subject vehicle and thereby exercises driving control.

The body ECU 17 is an electronic control unit that controls electric equipment of the subject vehicle. The body ECU 17 controls a direction indicator of the subject vehicle. The direction indicator is also referred to as turn signal lamp, turn lamp, or winker lamp. Further, the body ECU 17 can successively detect a reclining position of a seat of the subject vehicle. A reclining position can be detected from a rotation angle of a reclining motor. In the description of the present embodiment, a configuration in which a reclining position is detected by the body ECU 17 will be taken as an example but the present embodiment is not limited thereto. For example, the present embodiment may be so configured that a reclining position is detected by a seat ECU that adjusts an environment of a seat.

The interior camera 18 picks up an image of a predetermined range in the vehicle compartment of the subject vehicle. The interior camera 18 preferably picks up an image of a range embracing at least the driver's seat of the subject vehicle. The interior camera 18 more preferably picks up an image of a range embracing a passenger seat, and a rear seat in addition to the driver's seat of the subject vehicle. The interior camera 18 is comprised of, for example, a near infrared light source and a near infrared camera, a control unit that controls the light source and camera, and the like. The interior camera 18 is so configured that an image of an occupant of the subject vehicle irradiated with near infrared light from the near infrared light source is picked up with the near infrared camera. A captured image picked up with the near infrared camera is subjected to image analysis by a control unit. The control unit analyses the captured image to detect a feature amount of an occupant's face. The control unit may detect an orientation of the occupant's face, his/her degree of wakefulness, and the like based on the detected feature amount of the occupant's face. A degree of wakefulness can be detected from, for example, a degree of opening/closing of an eyelid.

The biosensor 19 measures bio-information of an occupant of the subject vehicle. The biosensor 19 successively outputs measured bio-information to the HCU 22. The present embodiment can be so configured that the biosensor 19 is provided in the subject vehicle. The present embodiment may also be so configured that the biosensor 19 is provided in a wearable device worn by an occupant. To provide the biosensor 19 in the subject vehicle, for example, a steering wheel, a seat, or the like can be used. In cases where the biosensor 19 is provided in a wearable device, the present embodiment can be so configured that a measurement result of the biosensor 19 is acquired by the HCU 22 through, for example, a short-range communication module. Examples of bio-information measured with the biosensor 19 are breath, pulse, heartbeat, and the like. The present embodiment may be so configured that what measures other bio-information than breach, pulse, and heartbeat is used as the biosensor 19. For example, the biosensor 19 may measure brain wave, heartbeat fluctuation, perspiration, body temperature, blood pressure, skin conductance, or the like.

The presentation device 20 is provided in the subject vehicle and presents information toward the interior of the subject vehicle. In other words, the presentation device 20 presents information to an occupant of the subject vehicle. The presentation device 20 presents information under the control of the HCU 22. The presentation device 20 includes, for example, a display device and a voice output device.

The display device makes a notification by displaying information. For the display device, for example, a meter MID (Multi Information Display), CID (Center Information Display), an indicator lamp, or HUD (Head-Up Display) can be used. The voice output device makes a notification by outputting voice. Examples of the voice output device include a speaker and the like.

The meter MID is a display device provided in front of the driver's seat in the vehicle compartment. For example, the present embodiment can be so configured that the meter MID is provided in a meter panel. The CID is a display device disposed in the center of the instrument panel of the subject vehicle. An example of the indicator lamp is a lamp that blinks for indicating a direction of lane change of the subject vehicle.

The HUD is provided in, for example, the instrument panel in the vehicle compartment. The HUD projects a display image formed by a projector onto a projection area defined in a front wind shield as a projection member. The light of an image reflected to the vehicle compartment side by the front wind shield is perceived by a driver seated in the driver's seat. As a result, the driver can view a virtual image of a display image formed ahead of the front wind shield, partly overlapped with the foreground. The HUD may be so configured as to project a display image onto a combiner provided in front of the driver's seat in place of the front wind shield.

The user input device 21 accepts an input from a user. An operating device that accepts an operation input from a user can be used as the user input device 21. The operating device may be a mechanical switch or may be a touch switch integrated with a display. The user input device 21 need not be an operating device that accepts an operation input as long as the device accepts an input from a user. For example, the user input device may be a voice input device that accepts a command input by voice from a user.

The HCU 22 is configured based on a computer including a processor, a volatile memory, a nonvolatile memory, I/O, and a bus connecting these items. The HCU 22 executes a control program stored in the nonvolatile memory and thereby performs varied processing related to interaction between an occupant and a system of the subject vehicle.

The blind mechanism 23 is a mechanism capable of changing an amount of natural light taken into the interior of the subject vehicle. The blind mechanism 23 varies an amount of natural light taken into the interior of the subject vehicle; the blind mechanism 23 can be so configured as to be provided on a window of the subject vehicle. The blind mechanism 23 can be so configured as to be provided on a front window, a rear window, or a side window of the subject vehicle. For the blind mechanism 23, for example, a dimming film capable of switching between a light transmissive state and a light shielded state by application of a voltage. For the blind mechanism 23, a mechanism that is in a light permissive state when not in operation and is a light shielded state when in operation can be adopted. The present embodiment may be so configured that any other material than a dimming film is used as the blind mechanism 23. For example, a mechanism that electrically closes a louver, a curtain, or the like and thereby changes an amount of natural light taken into the interior of the subject vehicle may be adopted.

The automated driving ECU 10 is configured based on a computer including a processor, a volatile memory, a nonvolatile memory, and a bus connecting these items. The automated driving ECU 10 executes a control program stored in the nonvolatile memory and thereby performs processing related to automated driving. This automated driving ECU 10 is equivalent to a control device for a vehicle. In the present embodiment, the automated driving ECU 10 is used in at least a vehicle capable of sleep-permitted automated driving. A configuration of the automated driving ECU 10 will be described in detail below.

### <General Configuration of Automated Driving ECU 10>

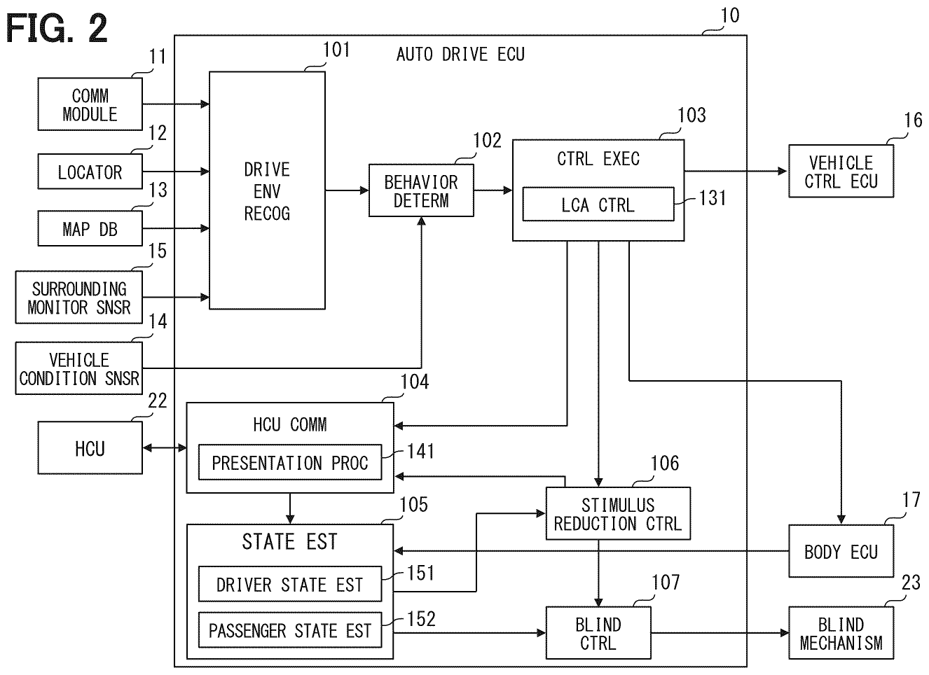

Subsequently, a description will be given to a general configuration of the automated driving ECU 10 with reference to FIG. 2. As shown in FIG. 2, the automated driving ECU 10 includes, as functional blocks, a travel environment recognition unit 101, a behavior determination unit 102, a control implementation unit 103, a HCU communication unit 104, a condition estimation unit 105, a stimulation reduction control unit 106, and a blind control unit 107. Execution of processing of each functional block of the automated driving ECU 10 by the computer is equivalent to execution of the control method for a vehicle. Part or all of the functions executed by the automated driving ECU 10 may be configured by hardware using one or more ICs or the like. Part or all of the functional blocks provided in the automated driving ECU 10 may be implemented by a combination of execution of software by the processor and a hardware member.

The travel environment recognition unit 101 recognizes a travel environment of the subject vehicle from a subject vehicle position acquired from the locator 12, map data acquired from the map DB 13, and sensing information acquired from the surroundings monitoring sensor 15. For example, the travel environment recognition unit 101 uses these pieces of information to recognize a position, a shape, and a moving state of an object in proximity to the subject vehicle and generates a virtual space reproducing the actual travel environment. With respect to a nearby vehicle of the subject vehicle, from sensing information acquired from the surroundings monitoring sensor 15, the travel environment recognition unit 101 can also recognize the presence, a relative position to the subject vehicle, a relative speed to the subject vehicle, and the like of the nearby vehicle as a travel environment. The travel environment recognition unit 101 can recognize a subject vehicle position on a map from the subject vehicle position and map data. In cases where positional information, speed information, or the like of a nearby vehicle can be acquired through the communication module 11, the travel environment recognition unit 101 can use also these pieces of information to recognize a travel environment.

Further, the travel environment recognition unit 101 can distinguish a manual driving area (hereafter, referred to as MD area) in a travel area of the subject vehicle. The travel environment recognition unit 101 can distinguish an automated driving area (hereafter, referred to as AD area) in a travel area of the subject vehicle. The travel environment recognition unit 101 can distinguish a ST section and non-ST section, described later, in an AD area from each other.

The MD area is an area where automated driving is prohibited. In other words, the MD area is an area where a driver is required to perform all of longitudinal direction control, lateral direction control, and surroundings monitoring in the subject vehicle. The longitudinal direction is a direction agreeing with the front and rear direction of the subject vehicle. The lateral direction is a direction agreeing with the width direction of the subject vehicle. The longitudinal direction control is equivalent to acceleration/deceleration control of the subject vehicle. The lateral direction control is equivalent to stewing control of the subject vehicle. For example, an ordinary road can be taken as an MD area. The MD area can also be defined as a traveling section of an ordinary road for which highly accurate map data has not been prepared.

The AD area is an area where automated driving is permitted. In other words, the AD area is an area where the subject vehicle can substitute with respect to one or more of longitudinal direction control, lateral direction control, and surroundings monitoring. For example, highway can be included in the AD area. The AD area can also be defined as a traveling section for which highly accurate map data has been prepared. For example, the automated driving of area limited LV 3 can be permitted only on a highway. The automated driving of congestion limited LV 3 is permitted only at a time of congestion in an AD area.

The AD area is divided into ST section and non-ST section. The ST section is a section where the automated driving of area limited LV 3 (hereafter, referred to as area limited automated driving) is permitted. The non-ST section is a section where the automated driving of LV 2 or lower level and the automated driving of congestion limited LV 3 can be performed. In the present embodiment, the non-ST section where the automated driving of LV 1 is permitted and the non-ST section where the automated driving of LV 2 is permitted are not separated from each other. A section that is not equivalent to an ST section in an AD area can be taken as a non-ST section.

The behavior determination unit 102 switches the control main body of a driving operation between a driver and a system of the subject vehicle. When the control of a driving operation is on the system side, the behavior determination unit 102 determines a traveling plan according to which the subject vehicle is run based on a result of recognition of a travel environment by the travel environment recognition unit 101. For the traveling plan, a route to a destination and a behavior the subject vehicle should take to arrive at the destination can be determined. Examples of behaviors include going straight, right turn, left turn, lane change, and the like.

Further, the behavior determination unit 102 changes an automation level of automated driving of the subject vehicle as required. The behavior determination unit 102 determines whether an automation level can be increased. For example, when the subject vehicle moves from an MD area to an AD area, it can be determined that a change from driving of LV 4 or lower levels to automated driving of LV 4 or higher level is possible. When it is determined that increase in automation level is possible and the increase in automation level is approved by a driver, the behavior determination unit 102 can increase the automation level.

When it is determined that reduction in automation level is required, the behavior determination unit 102 can reduce the automation level. Cases where it is determined that reduction in automation level is required are when an override is detected, a planned driving change time, and an unplanned driving change time. The override is an operation for a driver of the subject vehicle to voluntarily gain control of the subject vehicle. In other words, the override is the subject vehicle driver's intervention into operation. The behavior determination unit 102 can detect an override from sensing information acquired from the vehicle condition sensor 14. For example, the behavior determination unit 102 can detect an override when a steering torque detected with the steering torque sensor exceeds a threshold value. The behavior determination unit 102 can detect an override also when the accelerator sensor detects depression of the accelerator pedal. In addition, the behavior determination unit 102 can also detect an override when the brake sensor detects depression of the braked pedal. The planned driving change is a planned driving change according to a determination by a system. The unplanned driving change is an unplanned, sudden driving change according to a determination by a system.

When the control of a driving operation is on the side of a system of the subject vehicle, the control implementation unit 103 performs acceleration/deceleration control, steering control, and the like of the subject vehicle according to a traveling plan determined at the behavior determination unit 102 in cooperation with the vehicle control ECU 16. The control implementation unit 103 includes an LCA control unit 131 as a sub-functional block.

The LCA control unit 131 automatically causes a lane change. The LCA control unit 131 exercises LCA control to automatically causes a lane change from the present lane of the subject vehicle to an adjacent lane. In LCA control, a planned traveling path in such a shape that a target position of the present lane and the center of an adjacent lane are smoothly connected with each other is generated based on a result of recognition of a travel environment by the travel environment recognition unit 101. Then, a steering angle of the steering wheel of the subject vehicle is automatically controlled according to the planned traveling path and a lane change is thereby made from the present lane to the adjacent lane. When a peripheral situation meets a condition that allows lane change (hereafter, referred to as peripheral condition during automated driving of LV 4 or higher level, the LCA control unit 131 can start automatic lane change. During automated driving of LV 3 or lower levels, also on condition that a lane change request has been accepted from a driver through the user input device 21, the LCA control unit 131 can start automatic lane change.

Though a description is omitted in relation to the present embodiment for the sake of convenience, any other driving control as ACC (Adaptive Cruise Control) control or LTA (Lane Tracing Assist) control may be exercised aside from LCA control. The ACC control is control for implementing constant-speed traveling of the subject vehicle at a set speed or following traveling to a vehicle ahead. The LTA control is control for maintaining in-lane traveling of the subject vehicle. In LTA control, steering control is so exercised as to maintain in-lane traveling of the subject vehicle. To start a lane change in LCA control, LTA control can be temporarily stopped so that a departure from the present lane is possible. After completion of the lane change, LTA control can be resumed.

The HCU communication unit 104 performs processing of outputting information to the HCU 22 and processing of acquiring information from the HCU 22. The HCU communication unit 104 acquires a result of detection at the interior camera 18 and a result of measurement at the biosensor 19. The HCU communication unit 104 includes a presentation processing unit 141 as a sub-functional block. The presentation processing unit 141 indirectly controls information presentation at the presentation device 20.

The presentation processing unit 141 causes at least either of information presentation prompting surroundings monitoring from the presentation device 20 and information presentation notifying of a lane change being made at a planned lane change time when a lane change of the subject vehicle is planned at the LCA control unit 131. This planned lane change time is equivalent to a planned specific vehicle behavior change time. The information presentation prompting surroundings monitoring (hereafter, referred to as monitoring facilitating presentation) is a display, voice output, and the like prompting a driver to perform surroundings monitoring. Examples of monitoring facilitating presentation are a text display and voice output announcing “check the surroundings of your vehicle.” Information presentation notifying of a lane change being made (hereafter, referred to as lane change presentation) is, for example, blinking of an indicator lamp indicating a direction of lane change of the subject vehicle and the like. Hereafter, monitoring facilitating presentation and lane change presentation will be referred to as information presentation toward the interior. The presentation processing unit 141 is equivalent to a first vehicle-interior presentation control unit. At a planned lane change time, the body ECU 17 lights up a direction indicator for a direction to which a lane change is planned to be made.

The condition estimation unit 105 estimates a condition of an occupant of the subject vehicle. The condition estimation unit 105 estimates a condition of an occupant based on information acquired from the HCU 22 at the HCU communication unit 104 and information acquired from the body ECU 17. The condition estimation unit 105 includes a driver condition estimation unit 151 and a passenger condition estimation unit 152 as sub-functional blocks.

The driver condition estimation unit 151 estimates a condition of a driver of the subject vehicle. Processing at the driver condition estimation unit 151 is equivalent to a driver condition estimation step. The driver condition estimation unit 151 estimates at least whether a driver is in a sleep state. When a degree of wakefulness of a driver detected with the interior camera 18 is at a level corresponding to a sleep state, the driver condition estimation unit 151 can estimate that the driver is in a sleep state. When a result of measurement about a driver at the biosensor 19 is specific to a sleep state, the driver condition estimation unit 151 may estimate that the driver is in a sleep state. When a reclining position of a driver's seat acquired form the body ECU 17 is at a reclined angle at which a sleep state is estimated, the driver condition estimation unit 151 may estimate that the driver is in a sleep state. The present embodiment may be so configured that a reclining position of a driver's seat is acquired from the seat ECU.

When a degree of wakefulness of a driver detected with the interior camera 18 is at a level corresponding to a wakeful state, the driver condition estimation unit 151 can estimate that the driver is in a wakeful state. When a result of measurement about a driver at the biosensor 19 is not specific to a sleep state, the driver condition estimation unit 151 may estimate that the driver is in a wakeful state. When a reclining position of a driver's seat acquired from the body ECU 17 is not at a reclined angle at which a sleep state is estimated, the driver condition estimation unit 151 may be estimate that the driver is in a wakeful state. The driver condition estimation unit 151 may use also a detection result of a grasp sensor that detects whether a steering is grasped to estimate up to whether a driver estimated to be in a wakeful state grasps the steering.

The passenger condition estimation unit 152 estimates a condition of a passenger of the subject vehicle who is an occupant other than a driver of the subject vehicle. When a passenger exists, the passenger condition estimation unit 152 can estimate a condition of the passenger. Whether a passenger exists can be determined by the condition estimation unit 105 based on a seating sensor for other seats than a driver's seat or the like.

When a degree of wakefulness of a passenger detected with the interior camera 18 is at a level corresponding to a wakeful state, the passenger condition estimation unit 152 can estimate that the passenger is in a wakeful state. When a result of measurement about a passenger at the biosensor 19 is not specific to a sleep state, the passenger condition estimation unit 152 may estimate that the passenger is in a wakeful state. When a reclining position of a passenger's seat acquired from the body ECU 17 is not at a reclined angle at which a sleep state is estimated, the passenger condition estimation unit 152 may estimate that the passenger is in a wakeful state. The present embodiment may be so configured that a reclining position of a passenger's seat is also acquired from the seat ECU.

When a degree of wakefulness of a passenger detected with the interior camera 18 is at a level corresponding to a sleep state, the passenger condition estimation unit 152 can estimate that the passenger is in a sleep state. When a result of measurement about a passenger at the biosensor 19 is specific to a sleep state, the passenger condition estimation unit 152 may estimate that the passenger is in a sleep state. When a reclining position of a passenger's seat acquired from the body ECU 17 is at a reclined angle at which a sleep state is estimated, the passenger condition estimation unit 152 may estimate that the passenger is in a sleep state.

To estimate a condition of a driver at the HCU 22, the driver condition estimation unit 151 can acquire a result of estimation of the driver's condition at the HCU 22 to estimate the driver's condition. To estimate a condition of a passenger at the HCU 22, the passenger condition estimation unit 152 can acquire a result of estimation of the passenger's condition at the HCU 22 to estimate the passenger's condition.

When the driver condition estimation unit 151 estimates that a driver is in a sleep state during sleep-permitted automated driving of the subject vehicle, the stimulation reduction control unit 106 exercises control to reduce a stimulation to the driver. This processing at the stimulation reduction control unit 106 is equivalent to a stimulation reduction control step. As control to reduce a stimulation to a driver, the stimulation reduction control unit 106 exercises control to suppress at least either of monitoring facilitating presentation and lane change presentation (hereafter, referred to as information presentation suppression control) at a planned lane change time of the subject vehicle. That is, the stimulation reduction control unit exercises information presentation suppression control to suppress information presentation toward the interior. The stimulation reduction control unit 106 can, for example, give an instruction to the presentation processing unit 141 to exercise information presentation suppression control. Suppression of information presentation toward the interior may be refraining from performing information presentation toward the interior. Suppression of information presentation toward the interior may be performed by making the intensity of information presentation toward the interior lower than the intensity taken when the driver condition estimation unit 151 does not estimate that a driver is in a sleep state. Examples in which intensity is reduced in this case are reduction in the brightness of a display and reduction in the volume of voice output.

According to the above-mentioned configuration, when it is estimated that a driver is in a sleep state during sleep-permitted automated driving, control is exercised to suppress monitoring facilitating presentation and lane change presentation at a planned lane change time. Therefore, when a driver is in a sleep state at a planned lane change time during sleep-permitted automated driving, the sleep is less prone to be disturbed by a stimulation of information presentation to the driver. As a result, during automated driving during which a driver is permitted to sleep, the driver's convenience can be further enhanced.

When the passenger condition estimation unit 152 estimates that a passenger is in a wakeful state, the stimulation reduction control unit 106 does not preferably exercise information presentation suppression control even at a planned lane change time of the subject vehicle. According to the foregoing, in cases where a passenger is in a wakeful state, even when a driver is in a sleep state, information presentation toward the interior is performed as when a driver is not in a sleep state at a planned lane change time of the subject vehicle. Therefore, the passenger in a wakeful state can easily confirm monitoring facilitating presentation and lane change presentation and the passenger can get a feeling of security from automated driving.

When the driver condition estimation unit 151 estimates that a driver is not in a sleep state during sleep-permitted automated driving of the subject vehicle, the stimulation reduction control unit 106 does not preferably exercise information presentation suppression control. That is, the stimulation reduction control unit preferably prevents information presentation toward the interior from being suppressed. According to the foregoing, in cases where a driver is in a wakeful state, even during sleep-permitted automated driving, surroundings monitoring is prompted or a lane change being made is notified of; as a result, the driver can get a feeling of security from automated driving even if a lane change is made.

Even in cases where the driver condition estimation unit 151 estimates that a driver is not in a sleep state during sleep-permitted automated driving, when the driver condition estimation unit 151 estimates that the drive grasps a steering, the stimulation reduction control unit 106 may be so configured as to exercise information presentation suppression control. According to the foregoing, in cases where a driver highly possibly pays attention to driving during sleep-permitted automated driving of the subject vehicle, prompting surroundings monitoring or notification of a lane change being made can be suppressed to lessen irritation to the driver. Estimation that a driver grasps a steering at the driver condition estimation unit 151 can be performed based on a result of detection of a steering grasp senser or the like.

When a standby state is established at a planned lane change time of the subject vehicle, the stimulation reduction control unit 106 does not preferably exercise information presentation suppression control but preferably causes the presentation processing unit 141 to perform at least monitoring facilitating presentation as information presentation toward the interior. Meanwhile, when a standby state is not established at a planned lane change time of the subject vehicle, the stimulation reduction control unit 106 preferably exercises information presentation suppression control and suppresses at least monitoring facilitating presentation as information presentation toward the interior. In this case, information presentation suppression control is preferably control to prevent monitoring facilitating presentation. The standby state refers to a state in which the subject vehicle is caused to wait until a lane change become feasible. According to the foregoing, when a standby state is established, monitoring facilitating presentation is performed; thereby, an occupant can be made to perceive the present situation of standby state and be given a feeling of security from automated driving. Meanwhile, when a standby state is not established, a time for performing monitoring facilitating presentation is saved and a smooth lane change can be accordingly made. Further, since a time for performing monitoring facilitating presentation is saved, a lane change can be accordingly made with a leeway. Whether a standby state has been established can be determined by the LCA control unit 131 based on a result of recognition of a travel environment by the travel environment recognition unit 101 or the like. Whether a standby state has been established can also be determined by the behavior determination unit 102.

The blind control unit 107 controls the blind mechanism 23 and thereby increases or reduces an amount of natural light taken into the interior of the subject vehicle. When information presentation suppression control is not exercised at the stimulation reduction control unit 106 and at least monitoring facilitating presentation is performed as information presentation toward the interior at the presentation processing unit 141, the blind control unit 107 preferably prevents an amount of natural light taken into the interior of the subject vehicle from being reduced. According to the foregoing, when monitoring facilitating presentation is performed, it is possible to facilitate confirmation of the outside of the subject vehicle from the interior.

The blind control unit 107 may be capable of increasing or reducing an amount of natural light taken in of up to which window, front window, rear window, and side window, according to up to who of a driver and a passenger is estimated to be in a sleep state by the condition estimation unit 105. When all the occupants are in a sleep state, the blind control unit 107 can reduce an amount of natural light taken in at all of, for example, a front window, a rear window, and a side window as default.

### <Stimulation Reduction Related Processing at Automated Driving ECU 10>

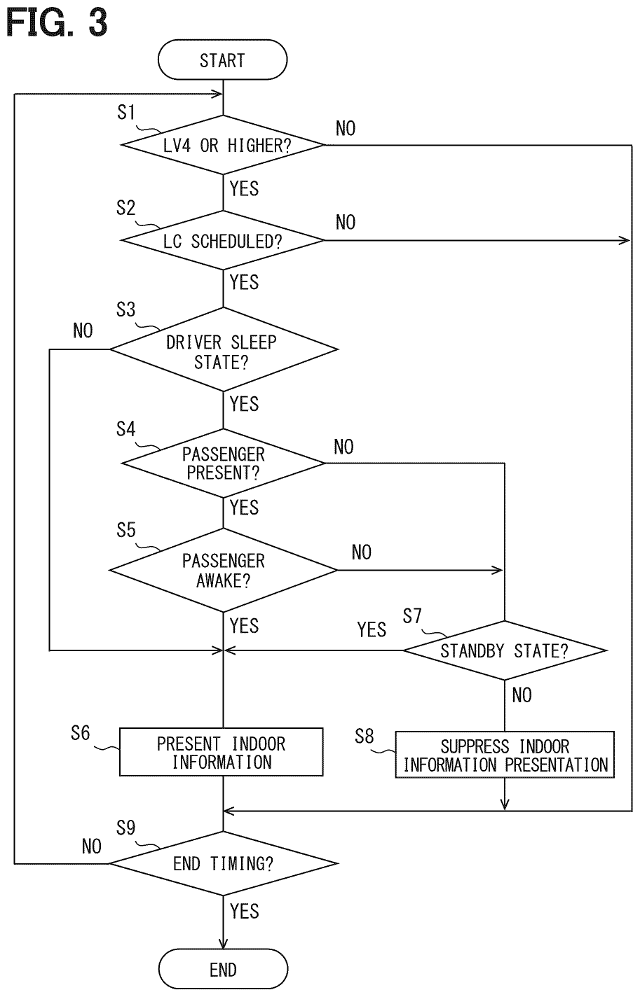

A description will be given to an example of a flow of processing related to control to reduce stimulation to a driver (hereafter, referred to as stimulation reduction related processing) at the automated driving ECU 10 with reference to the flowchart in FIG. 3. The flowchart in FIG. 3 can be so configured as to be started, for example, when a switch for starting an internal combustion engine or a motor generator of the subject vehicle (hereafter, referred to as power switch) is turned on.

When the subject vehicle is during automated driving of LV 4 or higher level at Step S1 (YES at S1), the processing proceeds to Step S2. That is, when the subject vehicle is during sleep-permitted automated driving, the processing proceeds to S2. Meanwhile, when the subject vehicle is during driving of a level of less than LV 4 (NO at S1), the processing proceeds to Step S9. The driving of a level of less than LV 4 also includes manual driving of LV 0. An automation level of the subject vehicle can be identified at the behavior determination unit 102.

When it is determined at Step 2 that the present time is a planned lane change time (YES at S2), the processing proceeds to Step S3. In the following drawings, a lane change is expressed as LC. Meanwhile, when the present time is not a planned lane change time (NO at S2), the processing proceeds to Step S9. Whether the present time is a planned lane change time can be determined at the LCA control unit 131.

When at Step S3, the driver condition estimation unit 151 estimates that a driver is in a sleep state (YES at S3), the processing proceeds to Step S4. Meanwhile, when at Step S3, the driver condition estimation unit 151 estimates that a driver is not in a sleep state (NO at S3), the processing proceeds to Step S6.

When at Step S4, a passenger exists (YES at S4), the processing proceeds to Step S5. When a passenger does not exist (NO at S4), the processing proceeds to Step S7. Whether a passenger exists can be estimated at the passenger condition estimation unit 152.

When at Step S5, the passenger condition estimation unit 152 estimates that a passenger is in a wakeful state (YES at S5), the processing proceeds to Step S6. Meanwhile, when the passenger condition estimation unit 152 estimates that a passenger is not in a wakeful state (NO at S5), the processing proceeds to Step S7. At Step S6, the presentation processing unit 141 causes information presentation toward the interior without suppression and the processing proceeds to Step S9.

When at Step S7, the subject vehicle is in a standby state (YES at S7), the processing proceeds to Step S6. Meanwhile, when the subject vehicle is not in a standby state (NO at S7), the processing proceeds to Step S8. Whether the subject vehicle is in a standby state can be determined at the LCA control unit 131. At Step S8, the stimulation reduction control unit 106 exercises information presentation suppression control to suppress information presentation toward the interior at the presentation processing unit 141 and the processing proceeds to Step S9.

When at Step S9, it is time to terminate the stimulation reduction related processing (YES at S9), the stimulation reduction related processing is terminated. Meanwhile, when it is not time to terminate the stimulation reduction related processing (NO at S9), the processing returns to S1 and is repeated. Examples of time to terminate stimulation reduction related processing are when a power switch of the subject vehicle is turned off and the like.

The present embodiment may be so configured that the processing of S4 to S5 in the flowchart in FIG. 3 is omitted. In this case, the present embodiment can be so configured that when a YES judgment is made at S3, the processing proceeds to S7. The present embodiment may be so configured that the processing of S7 in the flowchart in FIG. 3 is omitted. In this case, the present embodiment can be so configured that when a NO judgment is made at S4 and when a NO judgment is made at S5, the processing proceeds to S8. The present embodiment may be so configured that the processing of S4 to S5, and S7 in the flowchart in FIG. 3 is omitted. In this case, the present embodiment can be so configured that when a YES judgment is made at S3, the processing proceeds to S8.

### Second Embodiment

The present disclosure need not be configured as in the first embodiment and may be configured as in the second embodiment described below: Hereafter, a description will be given to an example of a configuration of the second embodiment with reference to the drawings.

<General Configuration of Vehicular System 1a>

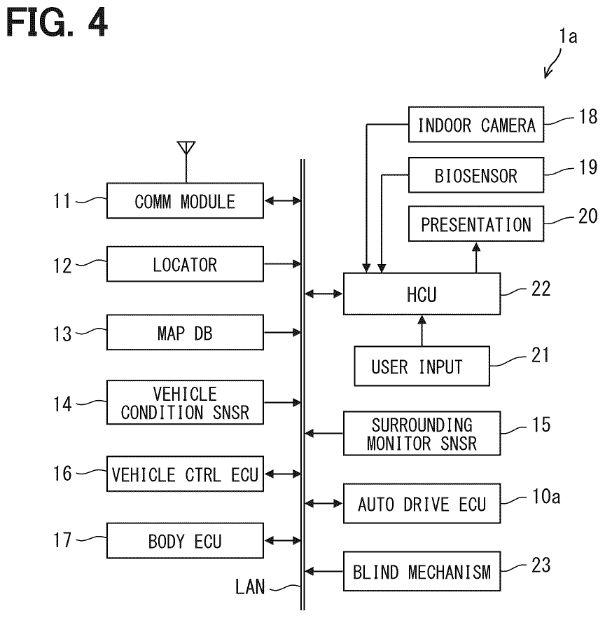

The vehicular system 1a shown in FIG. 4 can be used in an automated driving vehicle. As shown in FIG. 4, the vehicular system 1a includes: an automated driving ECU 10a, the communication module 11, the locator 12, the map DB 13, the vehicle condition sensor 14, the surroundings monitoring sensor 15, the vehicle control ECU 16, the body ECU 17, the interior camera 18, the biosensor 19, the presentation device 20, the user input device 21, the HCU 22, and the blind mechanism 23. The vehicular system 1a is identical with the vehicular system 1 in the first embodiment except that the automated driving ECU 10a is included in place of the automated driving ECU 10.

<General Configuration of Automated Driving ECU 10a>

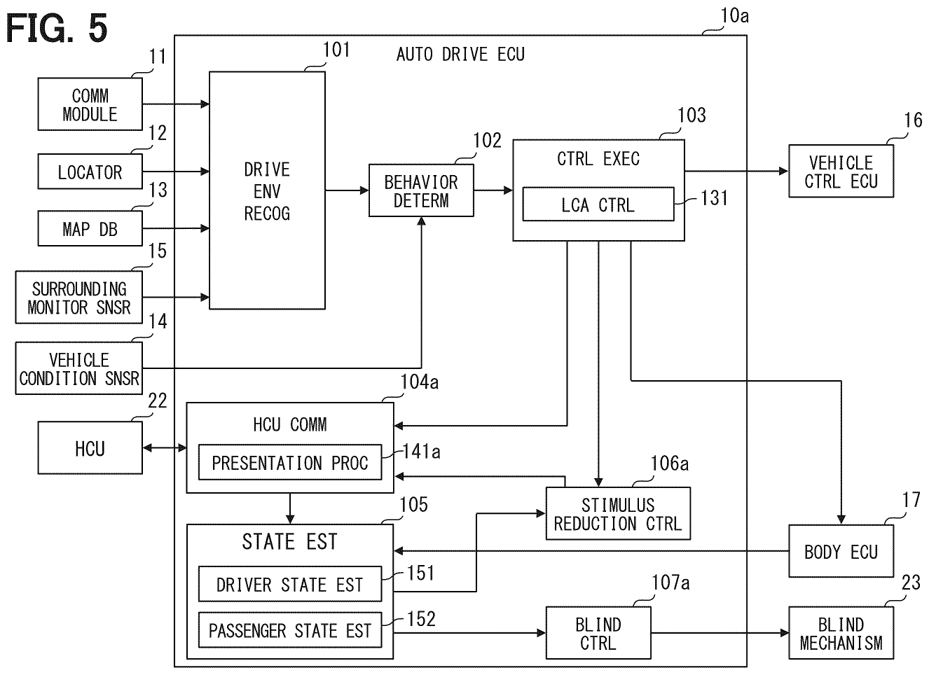

Subsequently, a description will be given to a general configuration of the automated driving ECU 10a with reference to FIG. 5. As shown in FIG. 5, the automated driving ECU 10a includes, as functional blocks, the travel environment recognition unit 101, the behavior determination unit 102, the control implementation unit 103, an HCU communication unit 104a, the condition estimation unit 105, a stimulation reduction control unit 106a, and a blind control unit 107a. The automated driving ECU 10a is identical with the automated driving ECU 10 in the first embodiment except that the HCU communication unit 104a, the stimulation reduction control unit 106a, and the blind control unit 107a are provided in place of the HCU communication unit 104, the stimulation reduction control unit 106, and the blind control unit 107. This automated driving ECU 10a is also equivalent to a control device for a vehicle. Execution of processing of each functional block of the automated driving ECU 10a by the computer is equivalent to execution of the control method for a vehicle.

The HCU communication unit 104a includes a presentation processing unit 141a as a sub-functional block. The HCU communication unit 104a is identical with the HCU communication unit 104 in the first embodiment except that the presentation processing unit 141a is provided in place of the presentation processing unit 141.

The presentation processing unit 141a causes at least the presentation device 20 to perform lane change presentation at a planned lane change time. As described in relation to the first embodiment, the lane change presentation is, for example, flickering of an indicator lamp that indicates a direction of lane change of the subject vehicle or the like. This lane change presentation is equivalent to in-vehicle presentation. The presentation processing unit 141a is equivalent to a second vehicle-interior presentation control unit. Though described in relation to the first embodiment as well, at a planned lane change time, the body ECU 17 lights up a direction indicator for a direction to which a lane change is planned to be made. This light-up of the direction indicator is equivalent to vehicle-exterior presentation.

When the driver condition estimation unit 151 estimates that a driver is in a sleep state during sleep-permitted automated driving of the subject vehicle, the stimulation reduction control unit 106a also exercises control to reduce a stimulation to the driver. This processing at the stimulation reduction control unit 106a is also equivalent to a stimulation reduction control step. The stimulation reduction control unit 106a exercises information presentation suppression control to at least suppress lane change presentation at a planned lane change time of the subject vehicle as control to reduce stimulation to a driver. Meanwhile, even when the driver condition estimation unit 151 estimates that a driver is in a sleep state during sleep-permitted automated driving of the subject vehicle, the stimulation reduction control unit 106a does not suppress light-up of a direction indicator for a direction to which a lane change is planned to be made at the body ECU 17. The stimulation reduction control unit 106a can, for example, give an instruction to the presentation processing unit 141a to exercise information presentation suppression control. Suppression of vehicle-interior presentation can be made by making the intensity of lane change presentation lower than the intensity taken when the driver condition estimation unit 151 does not estimate that a driver is in a sleep state. Examples in which intensity is reduced in this case are reduction in the brightness of a display and reduction in the volume of voice output.

According to the above-mentioned configuration, when it is estimated during sleep-permitted automated driving that a driver is in a sleep state, control to suppress lane change presentation is exercised at a planned lane change time. Therefore, when a driver is in a sleep state at a planned lane change time during sleep-permitted automated driving, the sleep is less prone to be disturbed by a stimulation of information presentation to the driver. As a result, during automated driving during which a driver is permitted to sleep, the driver's convenience can be further enhanced. Meanwhile, since light-up of a direction indicator toward the vehicle exterior is not suppressed, a driver of a nearby vehicle can be prevented from becoming difficult to recognize the subject vehicle's intention of lane change.

In cases where the passenger condition estimation unit 152 estimates that a passenger is in a wakeful state, even at a planned lane change time, the stimulation reduction control unit 106a does not preferably exercise information presentation suppression control. According to the foregoing, in cases where a passenger is in a wakeful state, even when a driver is in a sleep state, vehicle-interior presentation is performed as in cases where a driver is not in a sleep state at a planned lane change time of the subject vehicle. Therefore, a passenger in a wakeful state easily confirms lane change presentation and the passenger can get a feeling of security from automated driving.

When the driver condition estimation unit 151 estimates that a driver is not in a sleep state during sleep-permitted automated driving of the subject vehicle, the stimulation reduction control unit 106a does not preferably exercise information presentation suppression control. That is, vehicle-interior presentation is not preferably suppressed. According to the foregoing, in cases where a driver is awake even during sleep-permitted automated driving, a lane change being made can be notified of without reducing the intensity of information presentation; thus, a driver can get a feeling of securing from automated driving even when a lane change is made.

Even in cases where the driver condition estimation unit 151 estimates that a driver is not in a sleep state during sleep-permitted automated driving, when the driver condition estimation unit 151 estimates that the drive grasps a steering, the stimulation reduction control unit 106a may be so configured as to exercise information presentation suppression control. According to the foregoing, when a driver highly possibly pays attention to driving during sleep-permitted automated driving of the subject vehicle, irritation to the driver can be lessened by suppressing vehicle-interior presentation.

Regardless of whether information presentation suppression control is exercised at the stimulation reduction control unit 106, the blind control unit 107a is identical with the blind control unit 107 in the first embodiment except that the blind mechanism 23 is controlled.

<Stimulation Reduction Related Processing at Automated Driving ECU 10a>

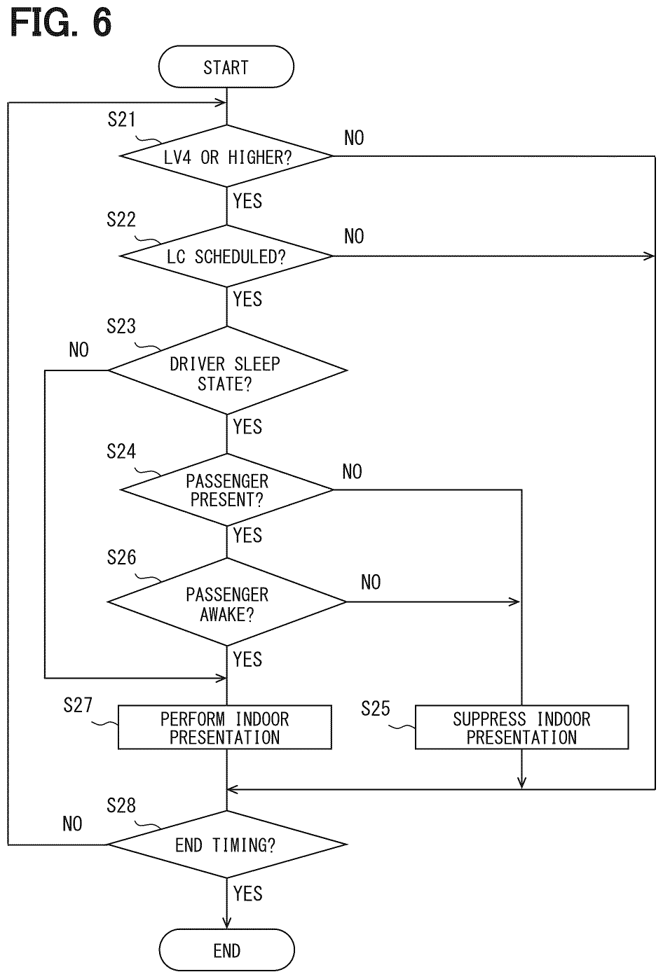

A description will be given to an example of a flow of stimulation reduction related processing at the automated driving ECU 10a with reference to the flowchart in FIG. 6. The flowchart in the FIG. 6 can be so configured as to be started, for example, when a power switch of the subject vehicle is turned on.

When at Step S21, the subject vehicle is during automated driving of LV 4 or higher level (YES at S21), the processing proceeds to Step S22. Meanwhile, when the subject vehicle is during driving of a level of less than LV 4 (NO at S21), the processing proceeds to Step S28. When it is determined at Step 22 that the present time is a planned lane change time (YES at S22), the processing proceeds to Step S23. Meanwhile, when the present time is not a planned lane change time (NO at S22), the processing proceeds to Step S28.

When at Step S23, the driver condition estimation unit 151 estimates that a drive is in a sleep state (YES at S23), the processing proceeds to Step S24. Meanwhile, when the driver condition estimation unit 151 estimates that a driver is not in a sleep state (NO at S23), the processing proceeds to Step S27. When at Step S24, a passenger exists (YES at S24), the processing proceeds to Step S26. Meanwhile, when a passenger does not exist (NO at S24), the processing proceeds to Step S25. At Step S25, the stimulation reduction control unit 106a exercises information presentation suppression control to suppress vehicle-interior presentation at the presentation processing unit 141a and the processing proceeds to Step S28.

When at Step S26, the passenger condition estimation unit 152 estimates that a passenger is in a wakeful state (YES at S26), the processing proceeds to Step S27. Meanwhile, when the passenger condition estimation unit 152 estimates that a passenger is not in a wakeful state (NO at S26), the processing proceeds to Step S25. At Step S27, the presentation processing unit 141a causes vehicle-interior presentation without suppression and the processing proceeds to Step S28.

When at Step S28, it is time to terminate the stimulation reduction related processing (YES at S28), the stimulation reduction related processing is terminated. Meanwhile, when it is not time to terminate the stimulation reduction related processing (NO at S28), the processing returns to S21 and is repeated. The present embodiment may be so configured that the processing of S24 to S25 in the flowchart in FIG. 6 is omitted. In this case, the present embodiment can be so configured that when a YES judgment is made at S23, the processing proceeds to S25.

### Third Embodiment

In the description related to the first and second embodiments, a configuration in which when a driver is estimated to be in a sleep state during sleep-permitted automated driving of the subject vehicle, a control to suppress information presentation at a planned lane change time has been taken as an example but the present disclosure need not be configured as mentioned above. For example, the present disclosure may be so configured that the stimulation reduction control unit 106, 106a exercise control to suppress information presentation at a planned time of behavior change, other than lane change, of the subject vehicle.

For example, the present disclosure may be so configured that when a driver is estimated to be in a sleep state during sleep-permitted automated driving of the subject vehicle, control is exercised to suppress information presentation at a planned time of acceleration at a certain or higher acceleration. In this case, the planned time of acceleration at a certain or higher acceleration is equivalent to a planned specific vehicle behavior change time. The present disclosure may be so configured that when a driver is estimated to be in a sleep state during sleep-permitted automated driving of the subject vehicle, control is exercised to suppress information presentation at a planned time of deceleration at a certain or higher deceleration. In this case, the planned time of deceleration at a certain or higher deceleration is equal to a planned specific vehicle behavior change time. The present disclosure may be so configured that when a driver is estimated to be in a sleep state during sleep-permitted automated driving of the subject vehicle, control is exercised to suppress information presentation at a planned time of turning at a certain or larger steering angle. In this case, the planned time of turning at a certain or larger steering angle is equal to a planned specific vehicle behavior change time.

Even with the above-mentioned configuration, when a driver is estimated to be in a sleep state during sleep-permitted automated driving of the subject vehicle, control is exercised to reduce stimulation to the driver by information presentation. Therefore, during automated driving during which a driver is permitted to sleep, the driver's convenience can be further enhanced.

### Fourth Embodiment

In the description of the above embodiments, a configuration in which the condition estimation unit 105 is provided with the passenger condition estimation unit 152 has been taken as an example but the present disclosure need not be configured as mentioned above. For example, the present disclosure may be so configured that the condition estimation unit 105 is not provided with the passenger condition estimation unit 152.

### Fifth Embodiment

In the description of the above embodiments, a configuration in which when a driver is estimated to be in a sleep state during sleep-permitted automated driving of the subject vehicle, control is exercised to suppress information presentation at a planned specific vehicle behavior change time has been taken as an example but the present disclosure need not be configured as mentioned above. For example, the present disclosure may be configured as in the fifth embodiment described below: Hereafter, a description will be given to an example of a configuration of the fifth embodiment with reference to the drawings.

<General Configuration of Vehicular System 1b>

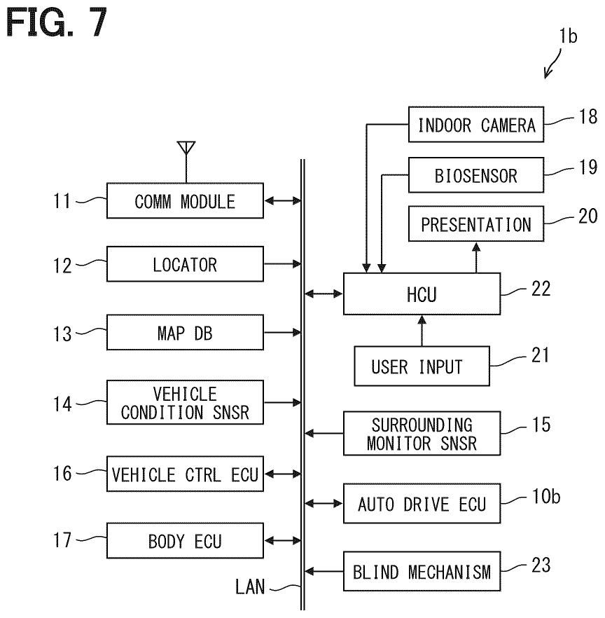

The vehicular system 1b shown in FIG. 7 can be used in an automated driving vehicle. As shown in FIG. 7, the vehicular system 1b includes: an automated driving ECU 10b, the communication module 11, the locator 12, the map DB 13, the vehicle condition sensor 14, the surroundings monitoring sensor 15, the vehicle control ECU 16, the body ECU 17, the interior camera 18, the biosensor 19, the presentation device 20, the user input device 21, the HCU 22, and the blind mechanism 23. The vehicular system 1b is identical with the vehicular system 1 in the first embodiment except that the automated driving ECU 10b is included in place of the automated driving ECU 10.

<General Configuration of Automated Driving ECU 10b>

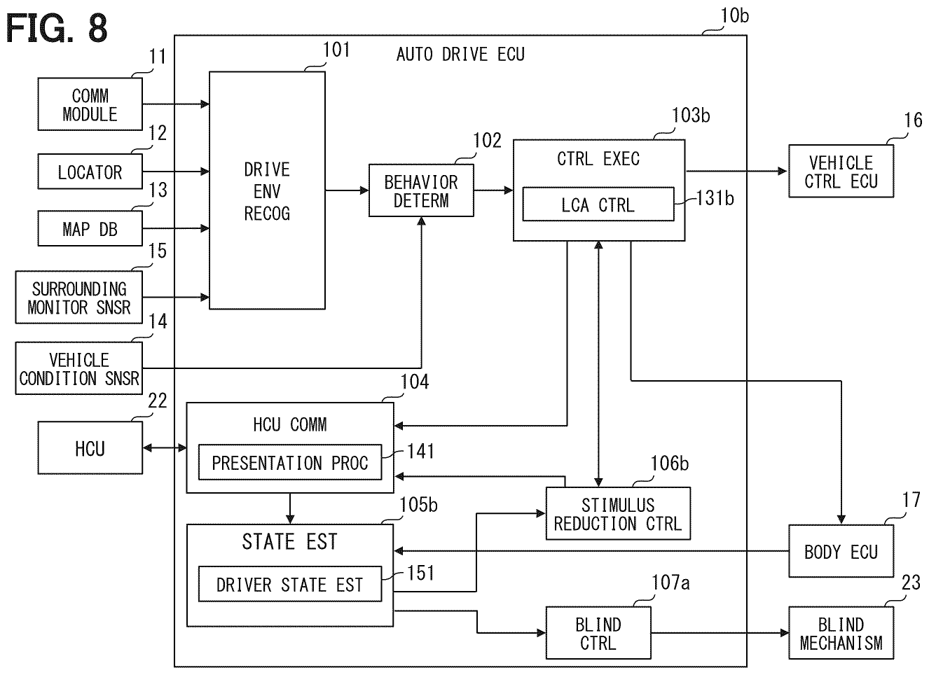

Subsequently, a description will be given to a general configuration of the automated driving ECU 10b with reference to FIG. 8. As shown in FIG. 8, the automated driving ECU 10b includes, as functional blocks, the travel environment recognition unit 101, the behavior determination unit 102, a control implementation unit 103b, the HCU communication unit 104, a condition estimation unit 105b, a stimulation reduction control unit 106b, and a blind control unit 107a. The automated driving ECU 10b is identical with the automated driving ECU 10 in the first embodiment except that the control implementation unit 103b, the condition estimation unit 105b, the stimulation reduction control unit 106b, and the blind control unit 107a are provided in place of the control implementation unit 103, the condition estimation unit 105, the stimulation reduction control unit 106, and the blind control unit 107. This automated driving ECU 10b is also equivalent to a control device for a vehicle. Execution of processing of each functional block of the automated driving ECU 10b by the computer is equivalent to execution of the control method for a vehicle. The blind control unit 107a is identical with the blind control unit 107a in the second embodiment.

The control implementation unit 103b includes an LCA control unit 131b as a sub-functional block. The control implementation unit 103b is identical with the control implementation unit 103 in the first embodiment except that the LCA control unit 131b is provided in place of the LCA control unit 131. The LCA control unit 131b is identical with the LCA control unit 131 in the first embodiment except that the former unit limits automatic lane change in accordance with an instruction from the condition estimation unit 105b.

The condition estimation unit 105b includes the driver condition estimation unit 151 as a sub-functional block. The condition estimation unit 105b is identical with the condition estimation unit 105 in the first embodiment except that the passenger condition estimation unit 152 is not provided.

When the driver condition estimation unit 151 estimates that a driver is in a sleep state during sleep-permitted automated driving of the subject vehicle, the stimulation reduction control unit 106b also exercises control to reduce stimulation to the driver. This processing at the stimulation reduction control unit 106b is also equivalent to a stimulation reduction control step. As control to reduce stimulation to a driver, the stimulation reduction control unit 106b exercises control to suppress a lane change dispensable to driving along a planned route to a destination during sleep-permitted automated driving (hereafter, referred to as unnecessary lane change). Control to suppress unnecessary lane change will be hereafter referred to as lane change suppression control. A destination set by an occupant of the subject vehicle through the user input device 21 can be taken as a destination in sleep-permitted automated driving. A destination in sleep-permitted automated driving may be a destination automatically estimated from a driving history of the subject vehicle by the automated driving ECU 10b. The stimulation reduction control unit 106b can exercise lane change suppression control, for example, by giving an instruction to the LCA control unit 131b.

As lane change suppression control, the stimulation reduction control unit 106b preferably exercises control to suppress at least lane change for overtake (hereafter, referred to as overtake suppression control). As lane change suppression control, in addition to overtake suppression control, the stimulation reduction control unit 106b may exercise control to suppress also a lane change for making way in order to let a following vehicle go ahead of the subject vehicle. The stimulation reduction control unit 106b can suppress unnecessary lane change by reducing a number of times or frequency of unnecessary lane change as compared with cases where unnecessary lane change is not suppressed. The stimulation reduction control unit 106b may suppress unnecessary lane change by refraining from making an unnecessary lane change.

According to the above-mentioned configuration, when a driver is estimated to be in a sleep state during sleep-permitted automated driving, control is exercised to suppress lane change dispensable to driving along a planned route to a destination in sleep-permitted automated driving. Therefore, when a driver is in a sleep state during sleep-permitted automated driving, the sleep is less prone to be disturbed by stimulation caused by a behavior change at a time of lane change dispensable to driving along a planned route to a destination in sleep-permitted automated driving. As a result, during automated driving during which a driver is permitted to sleep, the driver's convenience can be further enhanced.

When the driver condition estimation unit 151 estimates that a driver is not in a sleep state during sleep-permitted automated driving of the subject vehicle, the stimulation reduction control unit 106b does not preferably exercise lane change suppression control. According to the foregoing, in cases where a driver is awake even during sleep-permitted automated driving, the driver's stress can be lessened by giving high priority to smooth driving without exercising lane change suppression control.

Even when lane change suppression control is exercised, the stimulation reduction control unit 106b does not preferably suppress lane change for making way in order to let a following vehicle go ahead of the subject vehicle in a situation in which it is estimated that a traffic trouble should be avoided. An example of a situation in which it is estimated that a traffic trouble should be avoided is a case where a vehicle speed of a following vehicle is equal to or higher than a threshold value and a distance between the following vehicle and the subject vehicle is less than a specified value. According to the foregoing, even when lane change suppression control is exercised, way can be made for a tailgating following vehicle to avoid a traffic trouble.

<Stimulation Reduction Related Processing at Automated Driving ECU 10b>

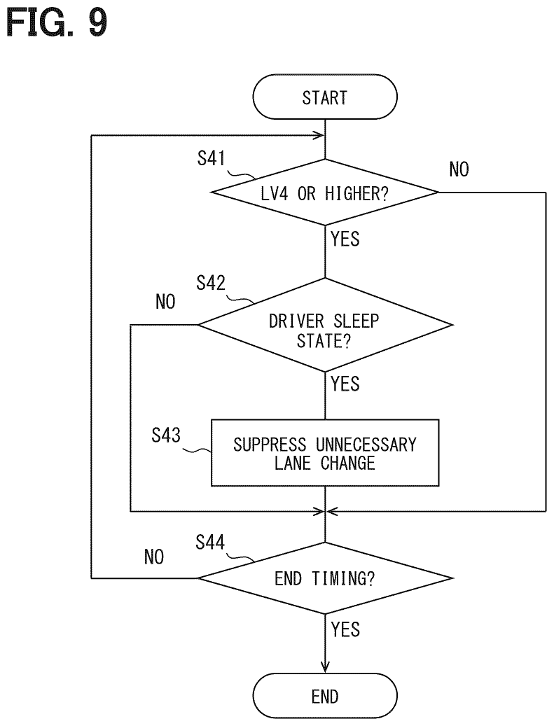

A description will be given to an example of a flow of stimulation reduction related processing at the automated driving ECU 10b with reference to the flowchart in FIG. 9. The flowchart in FIG. 9 can be so configured as to be started, for example, when a power switch of the subject vehicle is turned on.

When at Step S41, the subject vehicle is during automated driving of LV 4 or higher level (YES at S41), the processing proceeds to Step S42. Meanwhile, when the subject vehicle is during driving of a level of less than LV 4 (NO at S41), the processing proceeds to Step S44.

When at Step S42, the driver condition estimation unit 151 estimates that a driver is in a sleep state (YES at S42), the processing proceeds to Step S43. Meanwhile, when the driver condition estimation unit 151 estimates that the driver is not in a sleep state (NO at S42), the processing proceeds to Step S44. At Step S43, the stimulation reduction control unit 106b exercises lane change suppression control to suppress an unnecessary lane change at the LCA control unit 131b and the processing proceeds to Step S44.

As shown in FIG. 6, when at Step S26, the passenger condition estimation unit 152 estimates that a passenger is in a wakeful state (YES at S26), the processing proceeds to Step S27. Meanwhile, when the passenger condition estimation unit 152 estimates that a passenger is not in a wakeful state (NO at S26), the processing proceeds to Step S25. At Step S27, the presentation processing unit 141 causes vehicle-interior presentation without suppression and the processing proceeds to Step S28.

When at Step S44, it is time to terminate stimulation reduction related processing (YES at S44), the stimulation reduction related processing is terminated. Meanwhile, when it is not time to terminate the stimulation reduction related processing (NO at S44), the processing returns to S41 and is repeated.

### Sixth Embodiment

The present disclosure need not be configured as in the above-mentioned embodiments and may be configured as in the sixth embodiment described below. Hereafter, a description will be given to an example of a configuration of the sixth embodiment with reference to the drawings.

<General Configuration of Vehicular System 1c>

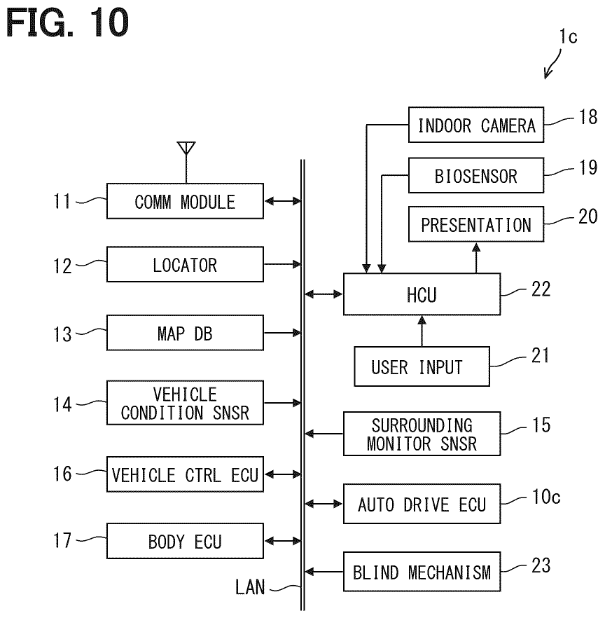

The vehicular system 1c shown in FIG. 10 can be used in an automated driving vehicle. As shown in FIG. 10, the vehicular system 1c includes: an automated driving ECU 10c, the communication module 11, the locator 12, the map DB 13, the vehicle condition sensor 14, the surroundings monitoring sensor 15, the vehicle control ECU 16, the body ECU 17, the interior camera 18, the biosensor 19, the presentation device 20, the user input device 21, the HCU 22, and the blind mechanism 23. The vehicular system 1c is identical with the vehicular system 1 in the first embodiment except that the automated driving ECU 10c is included in place of the automated driving ECU 10.

<General Configuration of Automated Driving ECU 10c>

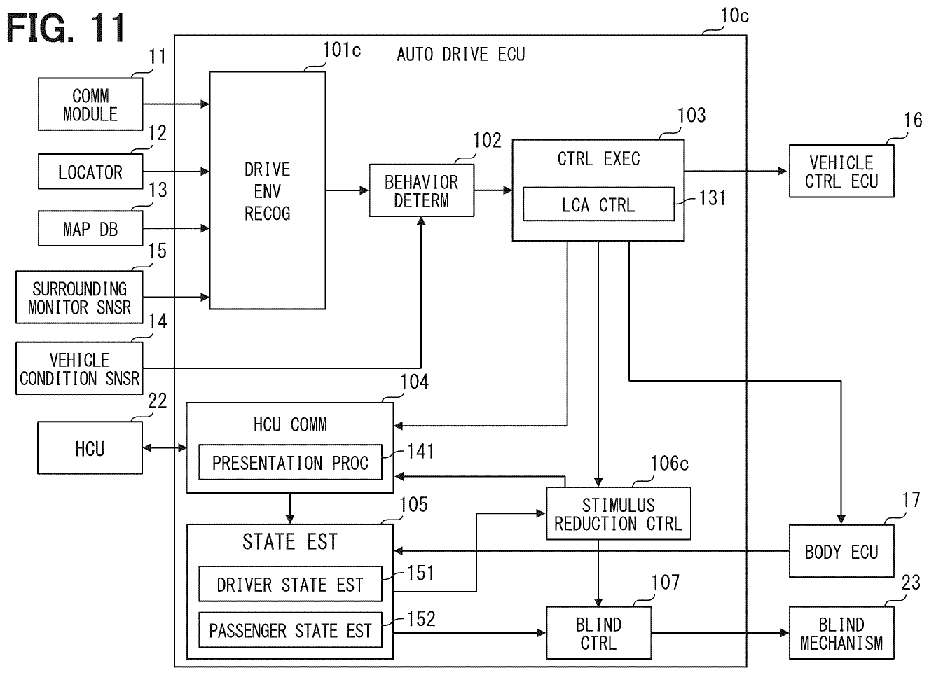

Subsequently, a description will be given to a general configuration of the automated driving ECU 1c with reference to FIG. 11. As shown in FIG. 11, the automated driving ECU 10c includes, as functional blocks, a travel environment recognition unit 101c, the behavior determination unit 102, the control implementation unit 103, the HCU communication unit 104, the condition estimation unit 105, a stimulation reduction control unit 106c, and the blind control unit 107. The automated driving ECU 10c includes the travel environment recognition unit 101c in place of the travel environment recognition unit 101. The automated driving ECU 10c includes the stimulation reduction control unit 106c in place of the stimulation reduction control unit 106. The automated driving ECU 10c is identical with the automated driving ECU 10 in the first embodiment except these respects. This automated driving ECU 10c is also equivalent to a control device for a vehicle. Execution of processing of each functional block of the automated driving ECU 10c by the computer is equivalent to execution of the control method for a vehicle.

The travel environment recognition unit 101c is identical with the travel environment recognition unit 101 in the first embodiment except that some processing is different. Hereafter, a description will be given to this difference. The travel environment recognition unit 101c determines whether the subject vehicle is traveling on a road dedicated to automated driving. The travel environment recognition unit 101c is equivalent to a travel condition determination unit. The travel environment recognition unit 101c can determine whether the subject vehicle is traveling on a road dedicated to automated driving according to whether a subject vehicle position on a map corresponds to a road dedicated to automated driving. In this case, the map DB 13 contains information about roads dedicated to automated driving. The road dedicated to automated driving refers to a road on which only automated driving vehicles can run. A road dedicated to automated driving may be some lane among a plurality of lanes. A road on which only automated driving vehicles during automated driving can run may be taken as a road dedicated to automated driving.

The stimulation reduction control unit 106c is identical with the stimulation reduction control unit 106 in the first embodiment except that some processing is different. Hereafter, a description will be given to this difference. When the travel environment recognition unit 101c determines that the subject vehicle is traveling on a road dedicated to automated driving, the stimulation reduction control unit 106c exercises control to reduce stimulation to an occupant of the subject vehicle. This is performed regardless of whether the condition estimation unit 105 estimates that an occupant of the subject vehicle is in a sleep state. The condition estimation unit 105e is equivalent to the occupant condition estimation unit.

The stimulation reduction control unit 106c is identical with the stimulation reduction control unit 106 in the first embodiment except that some processing is different. Hereafter, a description will be given to this difference. When the travel environment recognition unit 101c determines that the subject vehicle is traveling on a road dedicated to automated driving, the stimulation reduction control unit 106c exercises control to reduce stimulation to an occupant of the subject vehicle. This is performed regardless of whether the condition estimation unit 105 estimates that an occupant of the subject vehicle is in a sleep state. This processing at the stimulation reduction control unit 106c is also equivalent to a stimulation reduction control step. Hereafter, control to reduce stimulation to an occupant of the subject vehicle will be referred to as occupant stimulation reduction control. The occupant stimulation reduction control can be configured as the above-mentioned information presentation suppression control, lane change suppression control, and overtake suppression control as long as the control is to reduce stimulation given to a passenger as well as a driver. An occupant targeted here may be limited to a driver.

Since a vehicle other than automated driving vehicles does not run, accordingly, a less disturbance takes place on a road dedicated to automated driving than on other roads than roads dedicated to automated driving. Therefore, while the subject vehicle is traveling on a road dedicated to automated driving, it is less required for an occupant to pay attention to the driving of the subject vehicle. According to the configuration of the sixth embodiment, in such a situation that it is less required for an occupant to pay attention to the driving of the subject vehicle, stimulation to the occupant can be reduced regardless of whether the occupant is in a sleep state. As a result, in a situation in which it is less required for an occupant to pay attention to the driving of the subject vehicle, the occupant can be more relaxed.

### Seventh Embodiment

The present disclosure need not be configured as in the above-mentioned embodiments and may be configured as in the seventh embodiment described below. Hereafter, a description will be given to an example of a configuration of the seventh embodiment with reference to the drawings.

<General Configuration of Vehicular System 1d>

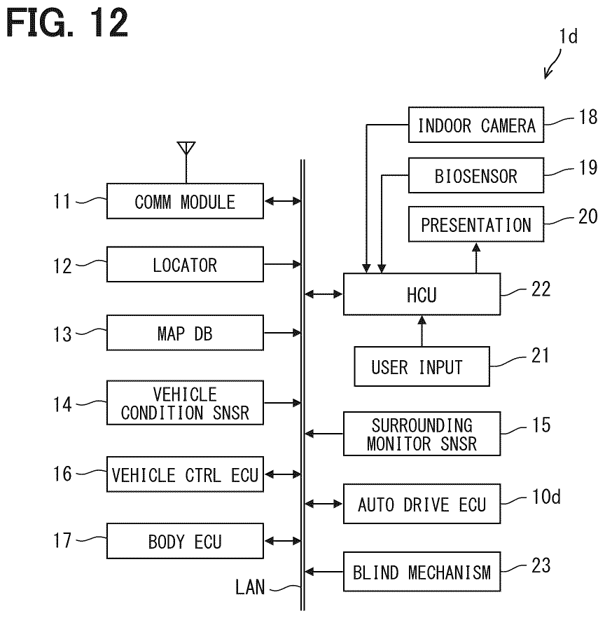

The vehicular system 1d shown in FIG. 12 can be used in an automated driving vehicle. As shown in FIG. 12, the vehicular system 1d includes: an automated driving ECU 10d, the communication module 11, the locator 12, the map DB 13, the vehicle condition sensor 14, the surroundings monitoring sensor 15, the vehicle control ECU 16, the body ECU 17, the interior camera 18, the biosensor 19, the presentation device 20, the user input device 21, the HCU 22, and the blind mechanism 23. The vehicular system 1d is identical with the vehicular system 1 in the first embodiment except that the automated driving ECU 10d is included in place of the automated driving ECU 10.

<General Configuration of Automated Driving ECU 10d>

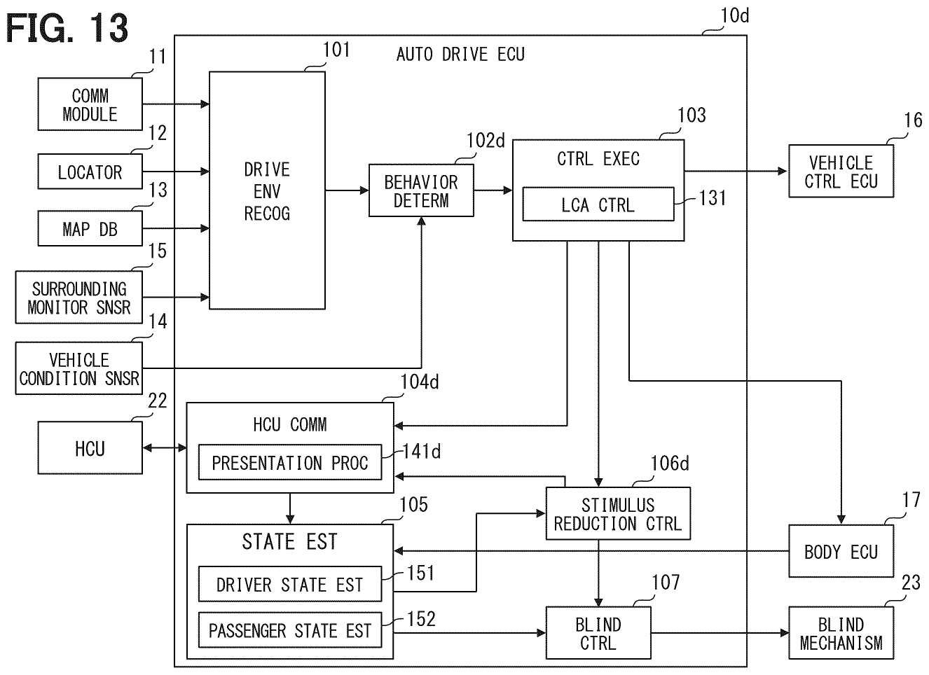

Subsequently, a description will be given to a general configuration of the automated driving ECU 10d with reference to FIG. 13. As shown in FIG. 13, the automated driving ECU 10d includes, as functional blocks, the travel environment recognition unit 101, a behavior determination unit 102d, the control implementation unit 103, an HCU communication unit 104d, the condition estimation unit 105, a stimulation reduction control unit 106d, and the blind control unit 107. The automated driving ECU 10d includes the behavior determination unit 102d in place of the behavior determination unit 102. The automated driving ECU 10d includes the HCU communication unit 104d in place of the HCU communication unit 104. The automated driving ECU 10d includes the stimulation reduction control unit 106d in place of the stimulation reduction control unit 106. The automated driving ECU 10d is identical with the automated driving ECU 10 in the first embodiment except these respects. This automated driving ECU 10d is also equivalent to a control device for a vehicle. Execution of processing of each functional block of the automated driving ECU 10d by the computer is equivalent to execution of the control method for a vehicle.

The behavior determination unit 102d is identical with the behavior determination unit 102 in the first embodiment except that some processing is different. Hereafter, a description will be given to this difference. The behavior determination unit 102d determines whether the subject vehicle should be brought into the above-mentioned standby state. That is, the behavior determination unit 102d determines whether the subject vehicle is in a standby state. The standby state is a state in which at a planned lane change time of the subject vehicle, the subject vehicle is caused to wait until a lane change becomes feasible. The lane change cited here refers to automatic lane change as mentioned above. Also hereafter, automatic lane change will be simply referred to as lane change. The behavior determination unit 102d can determine whether the subject vehicle is in a standby state based on a result of recognition of a travel environment by the travel environment recognition unit 101 and the like. When a nearby vehicle is detected within a certain range of a lane to which the subject vehicle is planned to make a lane change, the behavior determination unit 102d can determine that a standby state should be established. The certain range can be arbitrarily set. The behavior determination unit 102d successively determines whether the subject vehicle is in a standby state. As a result, the behavior determination unit 102d determines whether a standby state of the subject vehicle has lasted for a predetermined time. The predetermined time can be arbitrarily set. The behavior determination unit 102d is also equivalent to a travel condition determination unit.

The HCU communication unit 104d includes a presentation processing unit 141d as a sub-functional block. The HCU communication unit 104d is identical with the HCU communication unit 104 in the first embodiment except that the presentation processing unit 141d is provided in place of the presentation processing unit 141.

The presentation processing unit 141d is identical with the presentation processing unit 141 in the first embodiment except that some processing is different. Hereafter, a description will be given to this difference. When the behavior determination unit 102d determines that the subject vehicle is in a standby state, the presentation processing unit 141d causes at least the presentation device 20 to perform monitoring facilitating presentation and standby state presentation. That the subject vehicle is in a standby state can be determined by the behavior determination unit 102d. The monitoring facilitating presentation is the same information presentation promoting surroundings monitoring as described in relation to the first embodiment. The standby state presentation is information presentation notifying of the subject vehicle being in a standby state. As an example of standby state presentation, an image indicating that the subject vehicle cannot start a lane change can be displayed in the meter MID. Other examples of standby state presentation are text display, voice output, and the like announcing “Standby state.” A combination of the monitoring facilitating presentation and the standby state presentation is equivalent to standby related presentation. The presentation processing unit 141d is equivalent to a third vehicle-interior presentation control unit.

The stimulation reduction control unit 106d is identical with the stimulation reduction control unit 106 in the first embodiment except that some processing is different. Hereafter, a description will be given to this difference. When the behavior determination unit 102d determines that a standby state of the subject vehicle has passed for a predetermined time, the stimulation reduction control unit 106d causes standby related presentation again. Meanwhile, when the behavior determination unit 102d has not determined that a standby state of the subject vehicle has passed for a predetermined time, the stimulation reduction control unit 106d prevents standby related presentation from being caused again. According to the foregoing, when the subject vehicle is in a standby state, standby related presentation can be prevented from being frequently performed. Therefore, an occupant of the subject vehicle can be made less prone to feel irritation. This processing at the stimulation reduction control unit 106d is also equivalent to a stimulation reduction control step.

An occupant taken as a target of stimulation reduction at the stimulation reduction control unit 106d may be limited to a driver. The present disclosure may be so configured that whether the subject vehicle is in a standby state is determined by the travel environment recognition unit 101 or the control implementation unit 103.

### Eighth Embodiment

The present disclosure need not be configured as in the above-mentioned embodiments and may be configured as in the eighth embodiment described below. Hereafter, a description will be given to an example of a configuration of the eighth embodiment with reference to the drawings.

<General Configuration of Vehicular System 1e>

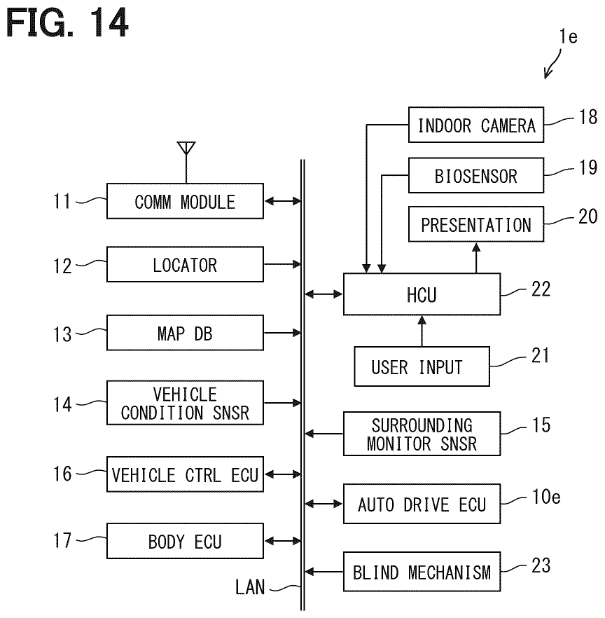

The vehicular system 1e shown in FIG. 14 can be used in an automated driving vehicle. As shown in FIG. 14, the vehicular system 1e includes: an automated driving ECU 10e, the communication module 11, the locator 12, the map DB 13, the vehicle condition sensor 14, the surroundings monitoring sensor 15, the vehicle control ECU 16, the body ECU 17, the interior camera 18, the biosensor 19, the presentation device 20, the user input device 21, the HCU 22, and the blind mechanism 23. The vehicular system 1e is identical with the vehicular system 1 in the first embodiment except that the automated driving ECU 10e is included in place of the automated driving ECU 10.

<General Configuration of Automated Driving ECU 10e>

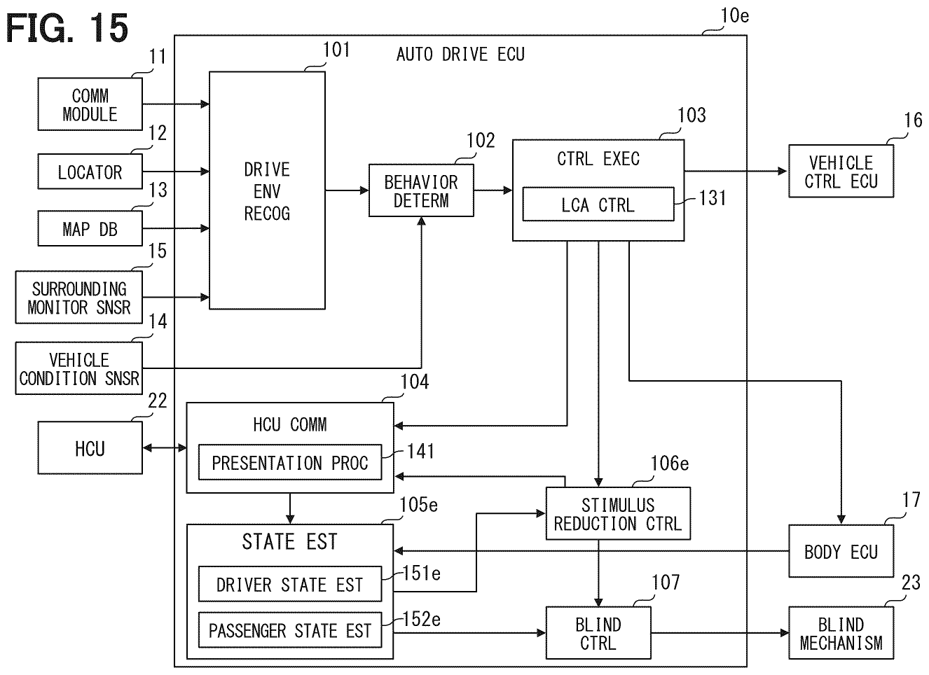

Subsequently, a description will be given to a general configuration of the automated driving ECU 10e with reference to FIG. 15. As shown in FIG. 15, the automated driving ECU 10e includes, as functional blocks, the travel environment recognition unit 101, the behavior determination unit 102, the control implementation unit 103, the HCU communication unit 104, a condition estimation unit 105e, the stimulation reduction control unit 106e, and the blind control unit 107. The automated driving ECU 10e includes the condition estimation unit 105e in place of the condition estimation unit 105. The automated driving ECU 10e includes the stimulation reduction control unit 106e in place of the stimulation reduction control unit 106. The automated driving ECU 10e is identical with the automated driving ECU 10 in the first embodiment except these respects. This automated driving ECU 10e is also equivalent to a control device for a vehicle. Execution of processing of each functional block of the automated driving ECU 10e by the computer is equivalent to execution of the control method for a vehicle.

The condition estimation unit 105e includes a driver condition estimation unit 151e and a passenger condition estimation unit 152e as sub-functional blocks. The driver condition estimation unit 151e is identical with the driver condition estimation unit 151 in the first embodiment except that some processing is different. The passenger condition estimation unit 152e is identical with the passenger condition estimation unit 152 in the first embodiment except that some processing is different. Hereafter, a description will be given to these differences.

The driver condition estimation unit 151e estimates whether a driver is performing a second task. The second task is an action other than driving permitted to a driver during automated driving free from a monitoring obligation as mentioned above. Examples of second tasks include viewing of such contents as videos, operation of a smartphone or the like, such actions as reading and taking a meal. The driver condition estimation unit 151e can estimate whether a driver is performing a second task from an image of the driver picked up with the interior camera 18. In this case, the driver condition estimation unit 151e can utilize a learning tool generated by machine learning. In addition, the driver condition estimation unit 151e may estimate whether a driver is performing a second task by referring to information of contents playback by the HCU 22. The driver condition estimation unit 151e can acquire contents playback information through the HCU communication unit 104.

The passenger condition estimation unit 152e estimates whether a passenger is performing an action equivalent to a second task. The action equivalent to a second task is an action identical with a second task except that the action is a passenger's action. The passenger condition estimation unit 152e can estimate whether a passenger is performing a second task from an image of the passenger picked up with the interior camera 18. The condition estimation unit 105e is also equivalent to the occupant condition estimation unit. An action equivalent a second task will be hereafter referred to as a second task equivalent action. A second task or a second task equivalent action will be hereafter referred to as target action.

The stimulation reduction control unit 106e is identical with the stimulation reduction control unit 106 in the first embodiment except that some processing is different. Hereafter, a description will be given to this difference. When the condition estimation unit 105e determines that a target action is being performed, the stimulation reduction control unit 106e exercises occupant stimulation reduction control. The condition estimation unit 105e determines that a target action is being performed is equivalent to a determination of that at least one of occupants is performing a target action. The occupant stimulation reduction control can be as described in relation to the sixth embodiment. This processing at the stimulation reduction control unit 106e is also equivalent to a stimulation reduction control step.

When a second task is disturbed while a driver is performing the second task, the driver's comfort is impaired. When a second task equivalent action is disturbed while a passenger is performing the second task equivalent action, the passenger's comfort is impaired. According to the configuration of the eighth embodiment, a second task and a second task equivalent action are made less prone to be disturbed by occupant stimulation reduction control. Therefore, an occupant's comfort is made less prone to be impaired.

When occupants can be distinguished to exercise occupant stimulation reduction control, the stimulation reduction control unit 106e may be configured as described below. The stimulation reduction control unit 106e can be so configured that occupant stimulation reduction control is exercised only to an occupant who is determined to be performing a target action. For example, this configuration is applicable to voice output by a directional speaker. Occupants taken as a target for stimulation reduction at the stimulation reduction control unit 106e may be limited to a driver.

### Ninth Embodiment

The present disclosure need not be configured as in the above-mentioned embodiments and may be configured as in the ninth embodiment described below. Hereafter, a description will be given to an example of a configuration of the ninth embodiment with reference to the drawings.

<General Configuration of Vehicular System 1f>

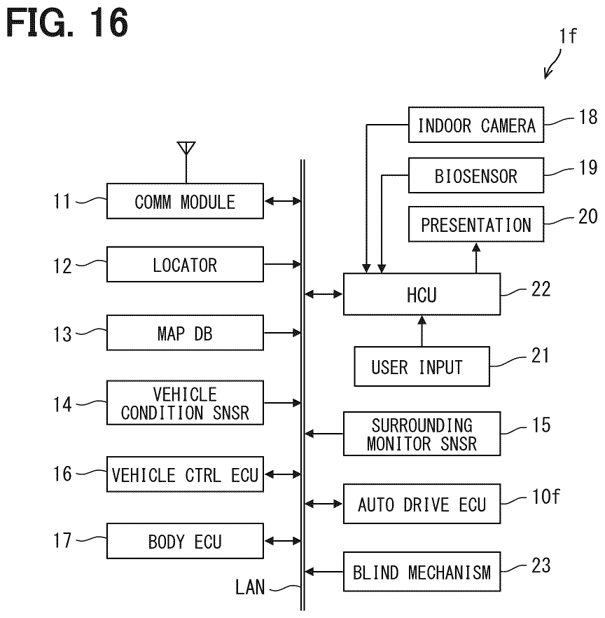

The vehicular system 1f shown in FIG. 16 can be used in an automated driving vehicle. As shown in FIG. 16, the vehicular system 1f includes: an automated driving ECU 10f, the communication module 11, the locator 12, the map DB 13, the vehicle condition sensor 14, the surroundings monitoring sensor 15, the vehicle control ECU 16, the body ECU 17, the interior camera 18, the biosensor 19, the presentation device 20, the user input device 21, the HCU 22, and the blind mechanism 23. The vehicular system 1f is identical with the vehicular system 1 in the first embodiment except that the automated driving ECU 10f is included in place of the automated driving ECU 10.

<General Configuration of Automated Driving ECU 10f>

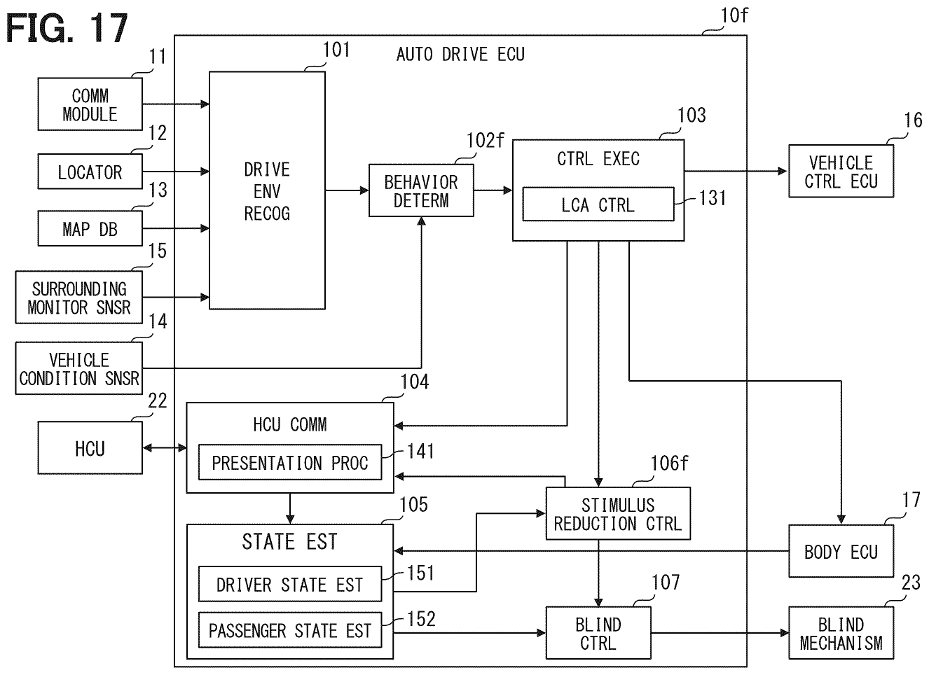

Subsequently, a description will be given to a general configuration of the automated driving ECU 10f with reference to FIG. 17. As shown in FIG. 17, the automated driving ECU 10f includes, as functional blocks, the travel environment recognition unit 101, a behavior determination unit 102f, the control implementation unit 103, the HCU communication unit 104, the condition estimation unit 105, a stimulation reduction control unit 106f, and the blind control unit 107. The automated driving ECU 10 includes the behavior determination unit 102f in place of the behavior determination unit 102. The automated driving ECU 10f includes the stimulation reduction control unit 106f in place of the stimulation reduction control unit 106. The automated driving ECU 10f is identical with the automated driving ECU 10 in the first embodiment except these respects. This automated driving ECU 10f is also equivalent to a control device for a vehicle. Execution of processing of each functional block of the automated driving ECU 10f by the computer is equivalent to execution of the control method for a vehicle.

The behavior determination unit 102f is identical with the behavior determination unit 102 in the first embodiment except that some processing is different. Hereafter, a description will be given to this difference. The behavior determination unit 102f determines a lane change of the subject vehicle. This lane change is automatic lane change. The behavior determination unit 102f can determine a lane change of the subject vehicle from a determined traveling plan. The behavior determination unit 102f distinguishes a lane change with overtake and a lane change without overtake from each other when determining a lane change. The behavior determination unit 102f is also equivalent to a travel condition determination unit. Hereafter, a lane change with overtake will be referred to as overtake lane change. Hereafter, a lane change without overtake will be referred to as non-overtake lane change.

The stimulation reduction control unit 106f is identical with the stimulation reduction control unit 106 in the first embodiment except that some processing is different. Hereafter, a description will be given to this difference. When a predetermined condition is met, the stimulation reduction control unit 106f exercises occupant stimulation reduction control. The occupant stimulation reduction control can be as described in relation to the sixth embodiment. The predetermined condition can be identical with, for example, a condition for reducing stimulation to driver at the stimulation reduction control unit 106, 106a, 106b. In this case, in occupant stimulation reduction control, control to reduce stimulation to a driver can be exercised. The predetermined condition may be identical with a condition for reducing stimulation to a driver at the stimulation reduction control unit 106c, 106d, 106e.

The stimulation reduction control unit 106f varies a degree of stimulation reduction in occupant stimulation reduction control between when an overtake lane change is determined and when a non-overtake lane change is determined. Whether a lane change is an overtake lane change or a non-overtake lane change is determined at the behavior determination unit 102f. In some cases, necessity for stimulation to an occupant may differ between overtake lane change and non-overtake lane change. According to the above-mentioned configuration, to cope with the foregoing, a degree of reduction of stimulation to occupants can be varied according to this necessity. This processing at the stimulation reduction control unit 106f is also equivalent to a stimulation reduction control step.

When a non-overtake lane change is determined, the stimulation reduction control unit 106f can increase a degree of stimulation reduction in occupant stimulation reduction control as compared with cases where an overtake lane change is determined. As compared with overtake lane change, an influence given to the lane change by a vehicle ahead of the subject vehicle is smaller in non-overtake lane change. Therefore, it is guessed that necessity for stimulation to occupants is smaller in non-overtake lane change than in overtake lane change. According to the above-mentioned configuration, therefore, even when occupant stimulation reduction control is exercised, a degree of reduction of stimulation to occupants can be more reduced with increase in necessity for stimulation to occupants in lane change.

When an overtake lane change is determined, the stimulation reduction control unit 106f is preferably configured as described below. The stimulation reduction control unit 106f preferably increases a degree of stimulation reduction in the second occupant stimulation reduction control as compared with the first of two-time lane changes for overtake.

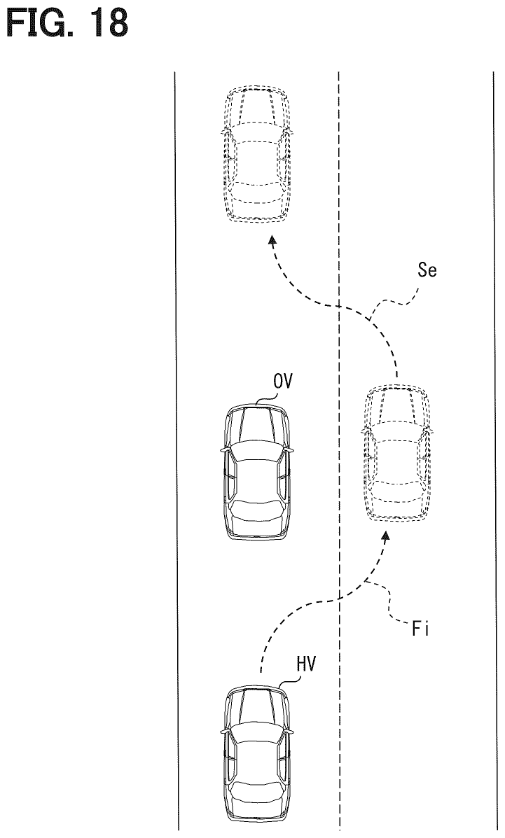

A description will be given to two-time lane changes for overtake with reference to FIG. 18. In FIG. 18, HV denotes the subject vehicle. In FIG. 18, OV denotes a vehicle ahead of the subject vehicle. The vehicle indicated by broken line in FIG. 18 represents the future subject vehicle in the overtake lane change. In FIG. 18, Fi denotes the first lane change. In FIG. 18, Se denotes the second lane change. As shown in FIG. 18, a lane change to a lane adjacent to the driving lane of the subject vehicle HV is the first lane change. A lane change returning from the adjacent lane to the original driving lane is the second lane change.

When the above-mentioned lane change presentation is performed in an overtake lane change, an occupant's consciousness is directed to presentation at the subject vehicle by performing presentation in the first lane change. Therefore, even if the presentation is lessened in the second lane change, the presentation is easily perceived. In general, a speed of a traveling vehicle is higher in an overtake lane than in a non-overtake lane. Therefore, it is guessed that necessity for an occupant to pay attention to the driving of the subject vehicle is lower in the second lane change than in the first lane change. According to the above-mentioned configuration, therefore, stimulation to an occupant with unnecessary intensity can be suppressed to enhance the occupant's comfort.

An occupant targeted for stimulation reduction at the stimulation reduction control unit 106f may be limited to a driver. The present disclosure may be so configured that whether the subject vehicle is to make an overtake lane change or a non-overtake lane change is determined at the travel environment recognition unit 101 or the control implementation unit 103.

### Tenth Embodiment

The present disclosure need not be configured as in the ninth embodiment and may be configured as in the tenth embodiment described below. Hereafter, a description will be given to an example of a configuration of the tenth embodiment. The tenth embodiment is identical in configuration with the ninth embodiment except that some processing at the stimulation reduction control unit 106f is different. Hereafter, a description will be given to this difference.

When an overtake lane change is determined, the stimulation reduction control unit 106f increases a degree of stimulation reduction in occupant stimulation reduction control as compared with cases where a non-overtake lane change is determined. Whether a lane change is an overtake lane change or a non-overtake lane change can be determined at the behavior determination unit 102f. In an overtake lane change, the subject vehicle overtakes a vehicle ahead and accordingly, a more disturbance takes place as compared with a non-overtake lane change. Therefore, in automated driving free from a monitoring obligation, a condition for starting can be made stricter in overtake lane change than in non-overtake lane change. In this case, it is guessed that necessity for an occupant to pay attention to the driving of the subject vehicle is lower in overtake lane change than in non-overtake lane change. According to the configuration of the tenth embodiment, to cope with the foregoing, an occupant can be more relaxed in a lane change in which necessity for an occupant to pay attention to the driving of the subject vehicle is lower.

### Eleventh Embodiment

The present disclosure need not be configured as in the above-mentioned embodiments and may be configured as in the eleventh embodiment described below. Hereafter, a description will be given to an example of a configuration of the eleventh embodiment with reference to the drawings.

<General Configuration of Vehicular System 1g>

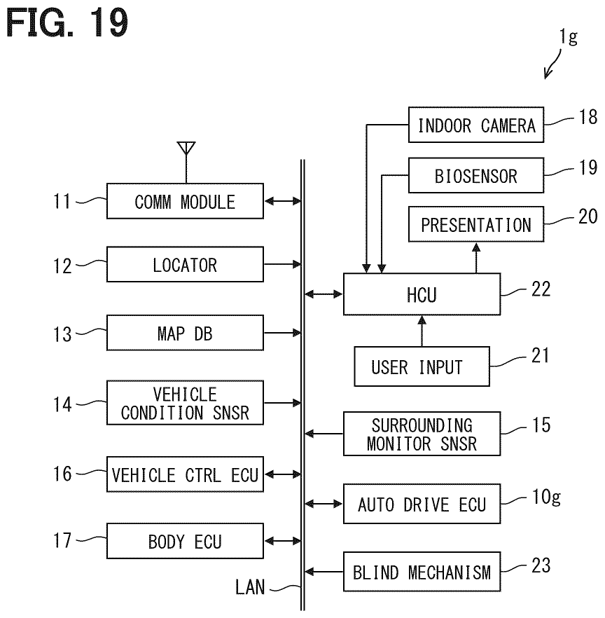

The vehicular system 1g shown in FIG. 19 can be used in an automated driving vehicle. As shown in FIG. 19, the vehicular system 1g includes: an automated driving ECU 10g, the communication module 11, the locator 12, the map DB 13, the vehicle condition sensor 14, the surroundings monitoring sensor 15, the vehicle control ECU 16, the body ECU 17, the interior camera 18, the biosensor 19, the presentation device 20, the user input device 21, the HCU 22, and the blind mechanism 23. The vehicular system 1g is identical with the vehicular system 1 in the first embodiment except that the automated driving ECU 10g is included in place of the automated driving ECU 10.

<General Configuration of Automated Driving ECU 10g>

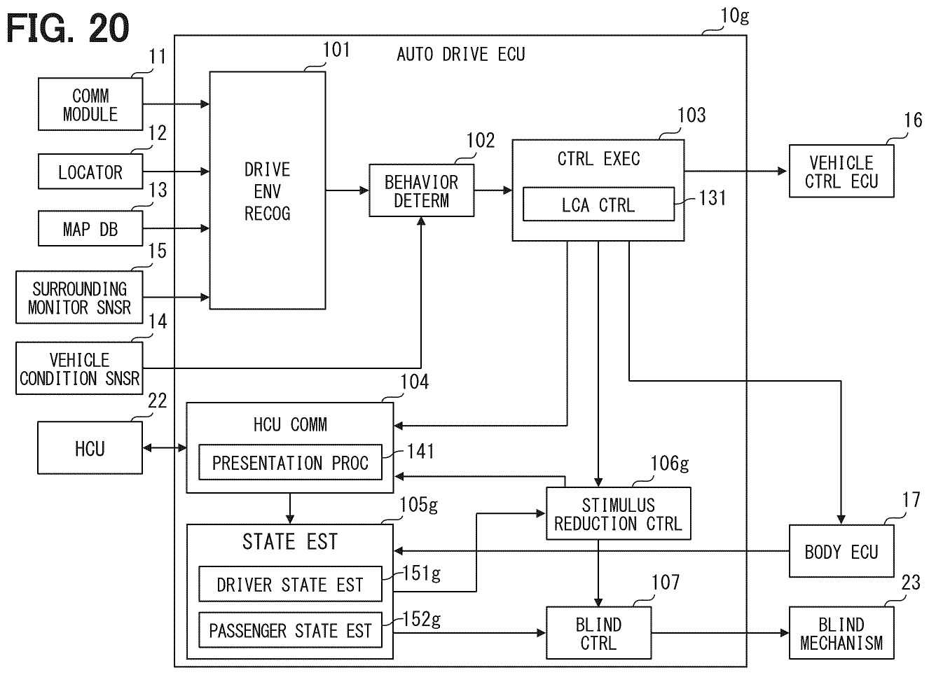

Subsequently, a description will be given to a general configuration of the automated driving ECU 10g with reference to FIG. 20. As shown in FIG. 20, the automated driving ECU 10g includes, as functional blocks, the travel environment recognition unit 101, behavior determination unit 102, control implementation unit 103, HCU communication unit 104, a condition estimation unit 105g, a stimulation reduction control unit 106g, and the blind control unit 107. The automated driving ECU 10g includes the condition estimation unit 105g in place of the condition estimation unit 105. The automated driving ECU 10g includes the stimulation reduction control unit 106g in place of the stimulation reduction control unit 106. The automated driving ECU 10g is identical with the automated driving ECU 10 in the first embodiment except these respects. This automated driving ECU 10g is also equivalent to a control device for a vehicle. Execution of processing of each functional block of the automated driving ECU 10g by the computer is equivalent to execution of the control method for a vehicle.

The condition estimation unit 105g includes a driver condition estimation unit 151g and a passenger condition estimation unit 152g as sub-functional blocks. The driver condition estimation unit 151g is identical with the driver condition estimation unit 151 in the first embodiment except that some processing is different. The passenger condition estimation unit 152g is identical with the passenger condition estimation unit 152 in the first embodiment except that some processing is different. Hereafter, a description will be given to these differences.

The driver condition estimation unit 151g estimates whether a driver is in a relaxed state. The driver condition estimation unit 151g can estimate whether a driver is in a relaxed state from an image of the driver picked up with the interior camera 18. In this case, the driver condition estimation unit 151g can utilize a learning tool generated by machine learning. In addition, when a reclining position of a driver's seat is at a reclined angle at which a relaxed state is estimated, the driver condition estimation unit 151g can estimate that the driver is in a relaxed state. A reclining position of the driver's seat can be acquired from the body ECU 17. The present embodiment may be so configured that a reclining position of a driver's seat is acquired from the seat ECU. When a configuration in which a relaxed state is estimated form a reclining position, the present disclosure may be so configured that a sleep state is not estimated from a reclining position.

The passenger condition estimation unit 152g estimate whether or not a passenger is in a relaxed state. The passenger condition estimation unit 152g can estimate whether a passenger is in a relaxed state from an image of the passenger picked up with the interior camera 18. The condition estimation unit 105g is also equivalent to the occupant condition estimation unit. When a reclining position of a passenger's seat is at a reclined angle at which a relaxed state is estimated, the passenger condition estimation unit 152g can estimate that the passenger is in a relaxed state.

The stimulation reduction control unit 106g is identical with the stimulation reduction control unit 106 in the first embodiment except that some processing is different. Hereafter, a description will be given to this difference. When it is estimated that all the occupants of the subject vehicle are in a sleep state or in a relaxed state, the stimulation reduction control unit 106g exercises control to prevent a notification about lane change from being made. This processing at the stimulation reduction control unit 106g is also equivalent to a stimulation reduction control step. That all the occupants of the subject vehicle are in a sleep state or in a relaxed state indicates that all the occupants of the subject vehicle are either in a sleep state or in a relaxed state. That all the occupants of the subject vehicle are in a sleep state or in a relaxed state can be determined at the condition estimation unit 105g. For example, control to prevent lane change presentation from being performed can be taken as control to prevent a notification about lane change from being made. This control is included in, for example, information presentation suppression control.

When all the occupants of the subject vehicle are in a sleep state or in a relaxed state, it is guessed that no occupant pays attention to the driving of the subject vehicle. In such a case, even though a notification is not made about lane change at a lane change time, an occupant is less prone to have a suspicion about the behavior of the subject vehicle. According to the configuration of the eleventh embodiment, in a situation in which an occupant is less prone to have a suspicion about the behavior of the subject vehicle, high priority can be given to the occupant's relaxation.

In relation to the eleventh embodiment, a configuration in which the condition estimation unit 105g determines an occupant's sleep state or relaxed state but the present disclosure need not be configured as mentioned above. For example, the present disclosure may be so configured that the condition estimation unit 105g determines only an occupant's sleep state of a sleep state and a relaxed state. In this case, when all the occupants of the subject vehicle are estimated to be in a sleep state, the stimulation reduction control unit 106g can exercise control to prevent a notification about lane change from being made.

### Twelfth Embodiment

The present disclosure need not be configured as in the above-mentioned embodiments and may be configured as in the twelfth embodiment described below. Hereafter, a description will be given to an example of a configuration of the twelfth embodiment with reference to the drawings.

<General Configuration of Vehicular System 1h>

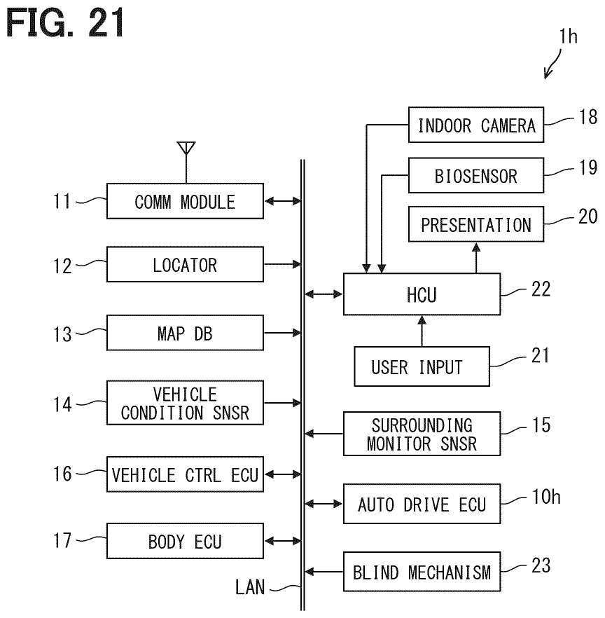

The vehicular system 1h shown in FIG. 21 can be used in an automated driving vehicle. As shown in FIG. 21, the vehicular system 1h includes: an automated driving ECU 10h, the communication module 11, the locator 12, the map DB 13, the vehicle condition sensor 14, the surroundings monitoring sensor 15, the vehicle control ECU 16, the body ECU 17, the interior camera 18, the biosensor 19, the presentation device 20, the user input device 21, the HCU 22, and the blind mechanism 23. The vehicular system 1h is identical with the vehicular system 1 in the first embodiment except that the automated driving ECU 10h is included in place of the automated driving ECU 10.

<General Configuration of Automated Driving ECU 10h>

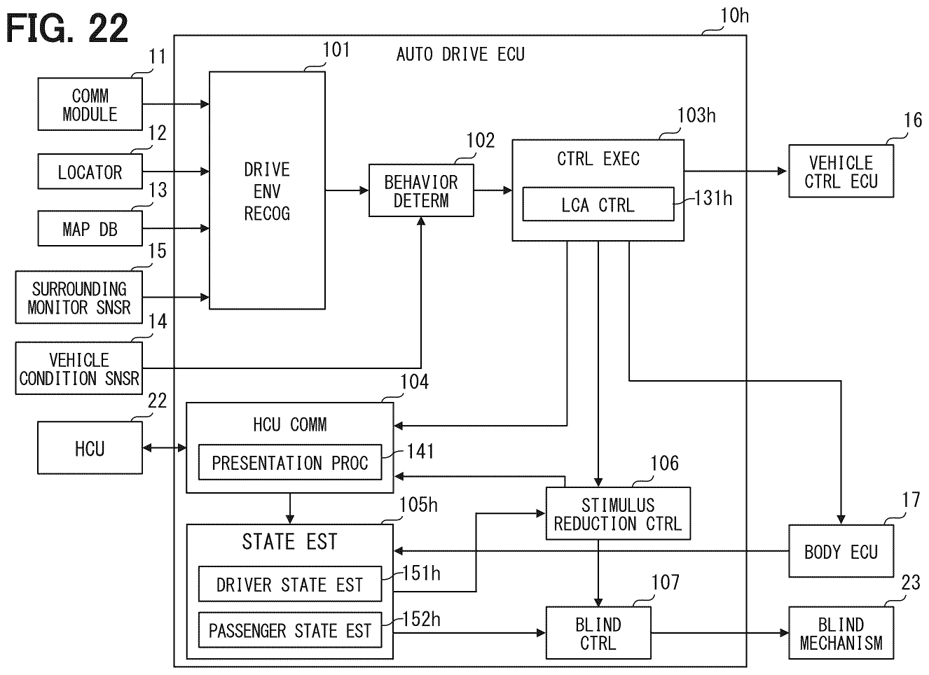

Subsequently, a description will be given to a general configuration of the automated driving ECU 10h with reference to FIG. 22. As shown in FIG. 22, the automated driving ECU 10h includes, as functional blocks, the travel environment recognition unit 101, behavior determination unit 102, a control implementation unit 103h, the HCU communication unit 104, a condition estimation unit 105h, the stimulation reduction control unit 106, and blind control unit 107. The automated driving ECU 10h includes the control implementation unit 103h in place of the control implementation unit 103. The automated driving ECU 10h includes the condition estimation unit 105h in place of the condition estimation unit 105. The automated driving ECU 10h is identical with the automated driving ECU 10 in the first embodiment except these respects. This automated driving ECU 10h is also equivalent to a control device for a vehicle. Execution of processing of each functional block of the automated driving ECU 10h by the computer is equivalent to execution of the control method for a vehicle.

The condition estimation unit 105h includes a driver condition estimation unit 151h and a passenger condition estimation unit 152h as sub-functional blocks. The driver condition estimation unit 151h is identical with the driver condition estimation unit 151 in the first embodiment except that some processing is different. The passenger condition estimation unit 152h is identical with the passenger condition estimation unit 152 in the first embodiment except that some processing is different. Hereafter, a description will be given to these differences.

The driver condition estimation unit 151h preferably estimates whether a driver is in a state in which it is unfavorable for acceleration in the lateral direction of the subject vehicle to be applied to the driver (hereafter, referred to as driver lateral G avoidance state). The acceleration in the lateral direction of the subject vehicle is so-called lateral G. Examples of driver lateral G avoidance state are car sickness, a state in which a driver is facing another occupant, and the like. A state implemented by turning of a seat or the like can be taken as a state in which a driver is facing another occupant. The driver condition estimation unit 151h can estimate whether a driver is in a driver lateral G avoidance state from an image of the driver picked up with the interior camera 18. In this case, the driver condition estimation unit 151h can utilize a learning tool generated by machine learning. In addition, the driver condition estimation unit 151h may estimate a driver lateral G avoidance state in which the driver is facing another occupant based on a state of turning of the driver's seat. A state of turning of the driver's seat can be acquired from the body ECU 17. The present disclosure may be so configured that a state of turning of the driver's seat is acquired from the seat ECU.

The driver condition estimation unit 151h preferably estimates a physical condition abnormal state of a driver of the subject vehicle. The physical condition abnormal state refers to a state in which a physical condition is abnormal, such as fainting. The driver condition estimation unit 151h can estimate whether a driver is in a physical condition abnormal state from an image of the driver picked up with the interior camera 18. The driver condition estimation unit 151h may estimate that a driver is in such a driver lateral G avoidance state as car sickness or in a physical condition abnormal state from bio-information of the driver measured with the biosensor 19.

The passenger condition estimation unit 152h determines whether a passenger is in a state in which it is unfavorable for acceleration in the lateral direction of the subject vehicle to be applied to the passenger (hereafter, referred to as passenger lateral G avoidance state). The same state as driver lateral G avoidance state can be taken as passenger lateral G avoidance state. In cases where the subject vehicle is such a passenger vehicle as a bus or a taxi, a seatbelt unworn state can also be included in the passenger lateral G avoidance state. The passenger condition estimation unit 152h can estimate a passenger lateral G avoidance state as the driver condition estimation unit 151 estimates a driver lateral G avoidance state. The passenger condition estimation unit 152h can estimate a seatbelt wearing state, for example, from an image of a driver picked up with the interior camera 18. Hereafter, driver lateral G avoidance state and passenger lateral G avoidance state will be collectively referred to as lateral G avoidance state.

The passenger condition estimation unit 152h preferably estimates a physical condition abnormal state of a passenger of the subject vehicle. The passenger condition estimation unit 152h can estimates whether a passenger is in a physical condition abnormal state from an image of the passenger picked up with the interior camera 18. The passenger condition estimation unit 152h may estimate that a driver is in such a driver lateral G avoidance state as car sickness or in a physical condition abnormal state from bio-information of the passenger measured with the biosensor 19.

The control implementation unit 103h includes an LCA control unit 131h as a sub-functional block. The control implementation unit 103h is identical with the control implementation unit 103 in the first embodiment except that the LCA control unit 131b is provided in place of the LCA control unit 131. The LCA control unit 131h is identical with the LCA control unit 131 in the first embodiment except that some processing is different. Hereafter, a description will be given to this difference.

The LCA control unit 131h changes a distance required from initiation to completion of a lane change at a lane change time of the subject vehicle according to a condition of an occupant of the subject vehicle estimated at the condition estimation unit 105h. Hereafter, a distance required from initiation to completion of a lane change at a lane change time of the subject vehicle will be referred to as lane change distance. The LCA control unit 131h can change a lane change distance, for example, by lengthening or shortening a distance in a planned traveling path at a lane change time. By changing a lane change distance, a lane change can be swiftly completed or lateral G applied to an occupant at a lane change time can be lessened. According to the above-mentioned configuration, therefore, a lane change with required behavior can be made according to a condition of an occupant. The LCA control unit 131h is equivalent to a lane change control unit.

When the condition estimation unit 105h estimates a lateral G avoidance state, the LCA control unit 131h preferably lengthens a lane change distance as compared with cases where a lateral G avoidance state is not estimated. When an occupant is in a lateral G avoidance state, it is more favorable for the occupant to lessen lateral G of the subject vehicle at a lane change time. According to the above-mentioned configuration, to cope with the foregoing, when an occupant is in a lateral G avoidance state, lateral G of the subject vehicle can be lessened at a lane change time. Therefore, the occupant's comfort can be enhanced.

When the condition estimation unit 105h estimates a physical condition abnormal state of an occupant, the LCA control unit 131h preferably shortens a lane change distance as compared with cases where a physical condition abnormal state is not estimated. When an occupant is in a physical condition abnormal state, it is preferable to swiftly make a lane change and move the subject vehicle to a refuge place. According to the above-mentioned configuration, to cope with the foregoing, when an occupant is in a physical condition abnormal state, a lane change can be swiftly completed. Examples of refuge places are road shoulder, service area, parking area, and the like. The present disclosure may be so configured that a physical condition abnormal state of an occupant estimated at the condition estimation unit 105h is limited to a driver's physical condition abnormal state.

### Thirteenth Embodiment

In relation to the above-mentioned embodiments, a configuration in which the automated driving ECU 10, 10a, 10b, 10c, 10d, 10e, 10f, 10g, 10h is provided with the blind control unit 107, 107a but the present disclosure need not be configured as mentioned above. For example, the present disclosure may be so configured that the automated driving ECU 10, 10a, 10b, 10c, 10d, 10e, 10f, 10g, 10h is not provided with the blind control unit 107, 107a. For example, the present disclosure may be so configured that the functions of the blind control unit 107, 107a are performed by the body ECU 17. The present disclosure may be so configured that the vehicular system 1, 1a, 1b, 1c, 1d, 1e, 1f, 1g, 1h does not include the blind control unit 107, 107a or the blind mechanism 23.

Note that the present disclosure is not limited to the embodiments described above and can variously be modified within the scope of the present disclosure. An embodiment obtained by appropriately combining the technical features disclosed in different embodiments may also be included in the technical scope of the present disclosure. The control device, control unit and the control method described in the present disclosure may be implemented by a special purpose computer which includes a processor programmed to execute one or more functions executed by computer programs. Alternatively, the device and the method thereof described in the present disclosure may also be implemented by a dedicated hardware logic circuit. Alternatively, the device and the method thereof described in the present disclosure may also be implemented by one or more dedicated computers configured as a combination of a processor executing a computer program and one or more hardware logic circuits. The computer program may be stored in a non-transitory tangible computer-readable recording medium as an instruction to be executed by a computer.

What is claimed is:

1. A vehicle control device used in a vehicle that performs sleep-permitted automated driving during which a driver is permitted to sleep, the vehicle control device comprising:
a driver condition estimation unit that is configured to estimate a condition of the driver; and
a stimulation reduction control unit that is configured to exercise control to reduce stimulation to the driver when the driver condition estimation unit estimates that the driver is in a sleep state during the sleep-permitted automated driving of the vehicle.

2. The vehicle control device according to claim 1, wherein
as the control to reduce stimulation to the driver, the stimulation reduction control unit exercises information presentation suppression control that is control to suppress information presentation at a planned specific vehicle behavior change time which is a planned time of specific vehicle behavior change of the vehicle.

3. The vehicle control device according to claim 2, further comprising:
a passenger condition estimation unit that is configured to estimate a condition of a passenger, other than the driver, of the vehicle,
wherein
when the passenger condition estimation unit estimates that the passenger is in a wakeful state, the stimulation reduction control unit does not exercise the information presentation suppression control even at the planned specific vehicle behavior change time.

4. The vehicle control device according to claim 2, further comprising:
a first vehicle-interior presentation control unit that is configured to cause information presentation toward an interior which is at least either of information presentation prompting surroundings monitoring and information presentation notifying of a lane change being made toward the interior of the vehicle at a planned automatic lane change time of the vehicle,
wherein
when the driver condition estimation unit estimates the driver is in a sleep state during the sleep-permitted automated driving of the vehicle, the stimulation reduction control unit exercises the information presentation suppression control to suppress the information presentation toward the interior by the first vehicle-interior presentation control unit as control to suppress information presentation at the planned specific vehicle behavior change time.

5. The vehicle control device according to claim 4, wherein
when the driver condition estimation unit estimates that the driver is not in a sleep state during the sleep-permitted automated driving of the vehicle, the stimulation reduction control unit does not exercise the information presentation suppression control to suppress the information presentation toward the interior.

6. The vehicle control device according to claim 4, wherein
the first vehicle-interior presentation control unit causes at least information presentation prompting surroundings monitoring as the information presentation toward the interior,
when a standby state in which the vehicle is caused to wait until a lane change becomes feasible is established at the planned automatic lane change time, the stimulation reduction control unit does not exercise the information presentation suppression control and causes the first vehicle-interior presentation control unit to perform the information presentation toward the interior, and
when the standby state is not established at the planned automatic lane change time, the stimulation reduction control unit exercises the information presentation suppression control to suppress the information presentation toward the interior.

7. The vehicle control device according to claim 4, further comprising:
a blind control unit that is configured to reduce an amount of natural light taken into the interior of the vehicle by controlling a blind mechanism capable of changing an amount of natural light taken into the interior of the vehicle,
wherein
the first vehicle-interior presentation control unit causes at least information presentation prompting surroundings monitoring as the information presentation toward the interior, and
when the stimulation reduction control unit does not cause the information presentation suppression control and causes the information presentation toward the interior, the blind control unit does not reduce an amount of natural light taken into the interior of the vehicle.

8. The vehicle control device according to claim 2, further comprising:
a second vehicle-interior presentation control unit that is configured to, at a planned automatic lane change time of the vehicle, causes vehicle-interior presentation as information presentation notifying of a lane change being made toward an interior of the vehicle,
wherein
when the driver condition estimation unit estimates that the driver is in a sleep state during the sleep-permitted automated driving of the vehicle, the stimulation reduction control unit exercises the information presentation suppression control to prevent vehicle-exterior presentation which is information presentation notifying an outside of the vehicle of a lane change being made from being suppressed and to cause the second vehicle-interior presentation control unit to perform the vehicle-interior presentation with lower intensity as compared with cases where the driver condition estimation unit does not estimate that the driver is in a sleep state.

9. The vehicle control device according to claim 1, wherein
the stimulation reduction control unit exercises lane change suppression control that is a control to suppress a lane change dispensable to traveling along a planned route to a destination in the sleep-permitted automated driving as control to reduce stimulation to the driver.

10. The vehicle control device according to claim 9, wherein
the stimulation reduction control unit exercises a control to suppress lane change for overtake as the lane change suppression control.

11. The vehicle control device according to claim 1, further comprising:
a travel condition determination unit that is configured to determine a travel condition of the vehicle; and
an occupant condition estimation unit that is configured to estimate a condition of an occupant of the vehicle,
wherein
when the travel condition determination unit determines that the vehicle is traveling on a road dedicated to automated driving, the stimulation reduction control unit exercises control to reduce stimulation to the occupant regardless of whether the occupant condition estimation unit estimates that the occupant is in a sleep state.

12. The vehicle control device according to claim 1, further comprising:
a travel condition determination unit that is configured to determine a travel condition of the vehicle; and
a third vehicle-interior presentation control unit that is configured to, when the travel condition determination unit determines that a standby state in which the vehicle is caused to wait until a lane change becomes feasible at a planned automatic lane change time of the vehicle, cause information presentation prompting surroundings monitoring and standby related presentation that is information presentation notifying of that the standby state has been established toward an interior of the vehicle,
wherein
when the travel condition determination unit determines that the standby state has lasted for a predetermined time, the stimulation reduction control unit causes the standby related presentation again, and
when it is not determined that the standby state has not lasted for the predetermined time, the stimulation reduction control unit does not cause the standby related presentation again.

13. The vehicle control device according to claim 1, further comprising:
an occupant condition estimation unit that is configured to estimate a condition of an occupant of the vehicle,
wherein
when the occupant condition estimation unit determines that at least one occupant is performing a second task or an action equivalent to the second task, an action other than driving, permitted for the driver during automated driving free from a monitoring obligation, the stimulation reduction control unit exercises a control to reduce stimulation to the occupant.

14. The vehicle control device according to claim 1, further comprising:
a travel condition determination unit that is configured to determine a travel condition of the vehicle,
wherein
the stimulation reduction control unit makes a degree of reduction for reducing stimulation to an occupant of the vehicle differ between a case where the travel condition determination unit determines an automatic lane change with overtake of the vehicle and a case where the travel condition determination unit determines an automatic lane change without overtake of the vehicle.

15. The vehicle control device according to claim 14, wherein
when the travel condition determination unit determines an automatic lane change without overtake of the vehicle, the stimulation reduction control unit increases the degree of reduction as compared with a case where the travel condition determination unit determines an automatic lane change with overtake of the vehicle.

16. The vehicle control device according to claim 14, wherein
when the travel condition determination unit determines an automatic lane change with overtake of the vehicle, the stimulation reduction control unit increases the degree of reduction in a second lane change as compared with a first of two-time lane changes for overtake.

17. The vehicle control device according to claim 1, further comprising:
an occupant condition estimation unit that is configured to estimate a condition of an occupant of the vehicle,
wherein
when all occupants of the vehicle are estimated to be in a sleep state or in a relaxed state, the stimulation reduction control unit exercises a control to prevent a notification about lane change from being made.

18. The vehicle control device according to claim 1, further comprising:
an occupant condition estimation unit that is configured to estimate a condition of an occupant of the vehicle; and
a lane change control unit that is configured to change a distance required from initiation to completion of a lane change at an automatic lane change of the vehicle according to a condition of the occupant estimated at the occupant condition estimation unit.

19. The vehicle control device according to claim 18, wherein
when a condition of an occupant in which it is unpreferable for acceleration in a lateral direction of the vehicle to be applied to the occupant is estimated, the lane change control unit lengthens a distance required from initiation to completion of a lane change at an automatic lane change time of the vehicle as compared with a case where the condition is not estimated.

20. A vehicle control method that is be used in a vehicle that performs sleep-permitted automated driving during which a driver is permitted to sleep,
the control method being performed by at least one processor,
the control method comprising:
a driver condition estimation step of estimating a condition of the driver; and
a stimulation reduction control step of, when it is estimated at the driver condition estimation step that the driver is in a sleep state during the sleep-permitted automated driving of the vehicle, exercising control to reduce stimulation to the driver.
